(19) United States
(12) Patent Application Publication
(10) Pub. No.: US 2010/0113445 A1
(43) Pub. Date: May 6, 2010

(54) Chemical Compounds

(75) Inventors: Felix Deanda, JR., Durham, NC (US); David Harold Drewry, Durham, NC (US); Paul Reid, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION

(21) Appl. No.: 12/531,760

(22) PCT Filed: Mar. 12, 2008
(86) PCT No.: PCT/US08/56622

§ 371 Date: Sep. 17, 2009

Related U.S. Application Data
(60) Provisional application No. 60/895,899, filed on Mar. 20, 2007.

(51) Int. Cl. A61K 31/5377; C07D 403/04; C07D 239/49; C07D 239/48; C07D 413/14; C07D 403/14; A61K 31/505; A61K 31/496; A61P 35/00

(52) U.S. Cl. 514/235.8; 544/324; 544/325; 544/296; 544/323; 544/122; 544/295; 514/275; 514/252.19

(57) ABSTRACT

The present invention relates to dianilinopyrimidine derivatives, compositions and medicaments containing the same, as well as processes for the preparation and use of such compounds, compositions and medicaments. Such dianilinopyrimidine derivatives are useful in the treatment of diseases associated with inappropriate Wee1 kinase activity.

### FIELD OF THE INVENTION

The present invention relates to dianilinopyrimidine derivatives that inhibit Wee1 kinase activity and methods for their use.

### BACKGROUND OF THE INVENTION

Protein kinases offer many opportunities for drug intervention, since phosphorylation is the most common post-translational modification (see, for example, Manning et al. (2002) Trends Biochem. Sci. 27(10):514-20). Protein kinases are key regulators of many cell processes, including signal transduction, transcriptional regulation, cell motility, and cell division. Kinase regulation of these processes is often accomplished by complex intermeshed kinase pathways in which each kinase is itself regulated by one or more other kinases. Aberrant or inappropriate protein kinase activity contributes to a number of pathological states including cancer, inflammation, cardiovascular and central nervous system diseases (see, for example, Wolf et al. (2002) Isr. Med. Assoc. J. 4(8):641-7; Li et al. (2002) J. Affect. Disord. 69(1-3):1-14; Srivastava (2002) Int. J. Mol. Med. 9(1):85-9; and Force et al. (2004) Circulation 109(10):1196-205). Due to their physiologic importance, variety, and ubiquity, protein kinases have become one of the most important and widely-studied family of enzymes in biochemical and medical research.

In mammalian cells there are several checkpoints in the cell cycle. The cell cycle arrests at these checkpoints if previous events (e.g. DNA replication or DNA repair) have not been completed. Progression through cell cycle checkpoints is regulated by the sequential activation and deactivation of a class of kinases known as cyclin-dependent kinases (Cdks). If a specific Cdk is not activated at the corresponding cell cycle checkpoint, the cell cycle will arrest at this checkpoint. When a cell cycle checkpoint is abrogated, uncontrolled cell proliferation can result.

Wee1 is a tyrosine kinase that plays a role in regulating the cell cycle in response to DNA damage. When DNA damage occurs, Wee1 halts progression from G2 into mitosis until DNA repair is complete. Wee1 arrests the cell cycle in G2 by phosphorylating the cyclin dependent kinase cdc2 to inactivate it. See, for example, Raleigh et al. (2000) J. Cell Sci. 113: 1727-36. When Wee1 is inhibited, the G2/M checkpoint is abrogated, inducing early cell division. Inhibition of Wee1 has been shown to kill cancer cells, possibly because the deregulated cell cycle progression that results from Wee1 inhibition damages cancer cells. See, for example Hashimoto et al. (2006) BMC Cancer 6:292. Thus, Wee1 kinase is a molecular target for the treatment of cancer.

Accordingly, there remains a need in the art for compounds that inhibit Wee1 kinase activity. Such compounds would be useful for treating diseases associated with aberrant Wee1 expression or activity.

### SUMMARY OF THE INVENTION

In one aspect of the present invention, there is provided a compound of Formula (I):

or a salt thereof, wherein:

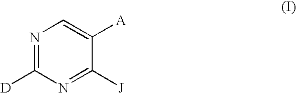

A is selected from —H, aryl optionally substituted with at least one R group, and heteroaryl optionally substituted with at least one R^{a} group;
Each R is independently selected from the group consisting of halo, OH, —NH₂, —CN, C₁-C₃ alkoxy, aryloxy, aralkoxy, CHO, —C(O)R″, —C(O)OR″, —C(O)OH, —C(O)H, —C(O)NR′R″, —NO₂, —N(H)C(O)R″, —N(H)S(O)₂R″, C₁-C₃ alkyl, C₁-C₃ hydroxyalkyl, C₁-C₃ haloalkyl, C₂-C₄ alkenyl, (CH₂)ₒX, —SR″, and aryl;
o is 0 or 1;
Each R^{a} is independently selected from the group consisting of C₁-C₆ alkyl, C₁-C₃ alkoxy, C(O)R″, and aralkyl;
J is selected from

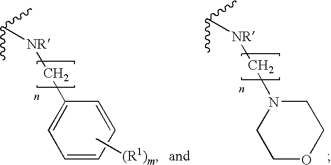

m is or 0 or 1;
n is 0, 1, or 2;
R¹ is halo, —CN, —NH₂, C₁-C₃ alkoxy, aryloxy, —C(O)N(H)R′, —C(O)OR″, heteroaryl optionally substituted with at least one C₁-C₃ alkyl, or (CH₂)_{q}X;
q is 0 or 1;

### D is:

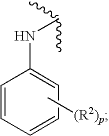

R² is selected from the group consisting of —O(CH₂)ₒNR′R″, —N(H)C(O)O(CH₂)ₒNR′R″, —(CH₂)ₒX, and —CH₂S(O)₂X;
p is 1;
o is 1 or 2;
R′ is —H or C₁-C₄ alkyl;
R″ is C₁-C₄ alkyl; and
X is heterocyclyl or heteroaryl.

In a second aspect of the present invention, there is provided a pharmaceutical composition comprising a therapeutically effective amount of a compound of formula (I) and one or more of pharmaceutically acceptable carriers, diluents and excipients.

In a third aspect of the present invention, there is provided a method of treating a disorder in a mammal, said disorder being mediated by inappropriate Wee1 activity, comprising: administering to said mammal a therapeutically effective amount of a compound of formula (I) or a salt thereof.

In a fourth aspect of the present invention, there is provided a method of treating cancer in a mammal comprising: administering to said mammal a therapeutically effective amount of a compound of formula (I) or a salt thereof.

In a fifth aspect of the present invention, there is provided a compound of formula (I), or a salt thereof for use in therapy.

In a sixth aspect of the present invention, there is provided the use of a compound of formula (I), or a salt thereof in the preparation of a medicament for use in the treatment of a disorder mediated by inappropriate Wee1 activity.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain monovalent hydrocarbon radical having from one to twelve carbon atoms. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₃ alkyl” and “C₁-C₆ alkyl” refer to an alkyl group, as defined above, containing at least 1, and at most 3 or 6 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, and n-hexyl.

As used herein, the term “alkylene” refers to a straight or branched chain divalent hydrocarbon radical having from one to ten carbon atoms. Examples of “alkylene” as used herein include, but are not limited to, methylene, ethylene, n-propylene, n-butylene, and the like.

As used herein, the term “C₁-C₃ alkylene” refers to an alkylene group, as defined above, which contains at least 1, and at most 3, carbon atoms respectively. Examples of “C₁-C₃ alkylene” groups useful in the present invention include, but are not limited to, methylene, ethylene, n-propylene, and isopropylene, and the like.

As used herein, the term “alkenyl” refers to a monovalent hydrocarbon radical having from two to ten carbons and at least one carbon-carbon double bond. Examples of “alkenyl” as used herein include, ethenyl, propenyl, 1-butenyl, 2-butenyl, and isobutenyl.

As used herein, the term “C₂-C₆ alkenyl” refers to an alkenyl group, as defined above, containing at least 2, and at most 6, carbon atoms. Examples of “C₂-C₆ alkenyl” groups useful in the present invention include, but are not limited to, ethenyl, propenyl, 1-butenyl, 2-butenyl, and isobutenyl.

As used herein, the term “halogen” refers to fluorine (F), chlorine (CI), bromine (Br), or iodine (I) and the term “halo” refers to the halogen radicals: fluoro (—F), chloro (—Cl), bromo (—Br), and iodo (—I).

As used herein, the term “C₁-C₃ haloalkyl” refers to an alkyl group as defined above containing at least 1, and at most 3 carbon atoms respectively substituted with at least one halo group, halo being as defined herein. Examples of such branched or straight chained haloalkyl groups useful in the present invention include, but are not limited to, methyl, ethyl, propyl, and isopropyl, substituted independently with one or more halos, e.g., fluoro, chloro, bromo and iodo.

As used herein, the term “heterocyclyl” refers to a monovalent three to twelve-membered non-aromatic heterocyclic ring, being saturated or having one or more degrees of unsaturation, containing one or more heteroatom ring substituents selected from S, S(O), S(O)₂, O, or N. Such a ring may be optionally fused to one or more other “heterocyclyl” ring(s) or cycloalkyl ring(s). Examples of “heterocyclyl” moieties include, but are not limited to, tetrahydrofuranyl, pyranyl, 1,4-dioxanyl, 1,3-dioxanyl, piperidinyl, piperazinyl, 2,4-piperazinedionyl, pyrrolidinyl, imidazolidinyl, pyrazolidinyl, morpholinyl, thiomorpholinyl, tetrahydrothiopyranyl, tetrahydrothiophenyl, and the like.

As used herein, the term “aryl” refers to a monovalent benzene ring or to a monovalent benzene ring system fused to one or more benzene or heterocyclyl rings to form, for example, anthracenyl, phenanthrenyl, napthalenyl, or benzodioxinyl ring systems. Examples of “aryl” groups include, but are not limited to, phenyl, 2-naphthyl, 1-naphthyl, biphenyl, and 1,4-benzodioxin-6-yl.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl-3-isoxazolylmethyl, and 2-imidazolyl ethyl.

As used herein, the term “heteroaryl” refers to a monovalent monocyclic five to seven membered aromatic ring, or to a fused bicyclic or tricyclic aromatic ring system comprising one, two, or three of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions. Examples of “heteroaryl” groups used herein include furanyl, thiophenyl, pyrrolyl, imidazolyl, pyrazolyl, triazolyl, tetrazolyl, thiazolyl, thienyl, oxazolyl, isoxazolyl, oxadiazolyl, oxo-pyridyl, quinoxalinyl, thiadiazolyl, isothiazolyl, pyridyl, pyridazyl, pyrazinyl, pyrimidyl, quinazolinyl, quinolinyl, isoquinolinyl, benzofuranyl, benzothiophenyl, indolyl, benzodioxol, pyrrolopyridyl, pyrrolopyrimidyl, and indazolyl.

In some embodiments of the present invention, the heteroaryl group is a C₂-C₉ heteroaryl group. As used herein, the term “C₂-C₉ heteroaryl” refers to an alkenyl group, as defined above, containing at least 2 and at most 9 carbon atoms.

As used herein, the term “alkoxy” refers to the group RₐₗₖO—, where Rₐₗₖ is alkyl as defined above and the term “C₁-C₃ alkoxy” refers to an alkoxy group as defined herein wherein the alkyl moiety contains at least 1, and at most 3 carbon atoms. Exemplary “C₁-C₃ alkoxy” groups useful in the present invention include, but are not limited to, methoxy, ethoxy, n-propoxy, and isopropoxy.

As used herein the term “aralkoxy” refers to the group R_{b}RₐO—, where Rₐ is alkylene and R_{b} is aryl or heteroaryl all as defined above. In some embodiments, the aralkoxy group contains 1 to 3 carbon atoms in the alkoxy moiety. In certain embodiments, the aralkoxy contains 1 carbon atom in the alkoxy moiety.

As used herein the term “aryloxy” refers to the group RₐO—, where Rₐ is aryl as defined above.

As used herein, the term “hydroxyalkyl” refers to an alkyl group as defined above substituted with at least one —OH. Examples of branched or straight chained C₁₋₄ hydroxyalkyl groups useful in the present invention include, but are not limited to, methyl, ethyl, propyl, isopropyl, substituted independently with one or more —OH such as hydroxymethyl, hydroxyalkyl, hydroxypropyl, and hydroxyisopropyl, hydroxyisobutyl, hydroxyl-n-butyl, and hydroxyl-t-butyl.

As used herein, the term “optionally” means that the subsequently described event(s) may or may not occur, and includes both event(s), which occur, and events that do not occur.

As used herein, the term “substituted” refers to substitution with the named substituent or substituents, multiple degrees of substitution being allowed unless otherwise stated.

The present invention includes solvates of the disclosed compounds and salts. As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include, without limitation, water, ethanol and acetic acid. In one embodiment, the solvent used is water.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

In one aspect of the present invention, there is provided a compound of Formula (I):

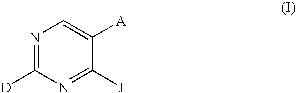

wherein:
A is selected from —H, aryl optionally substituted with at least one R group, and heteroaryl optionally substituted with at least one R^{a} group;
Each R is independently selected from the group consisting of halo, —OH, —NH₂, —CN, C₁-C₃ alkoxy, aryloxy, aralkoxy, —CHO, —C(O)R″, —C(O)OR″, —C(O)OH, —C(O)H, —C(O)NR′R″, —NO₂, —N(H)C(O)R″, —N(H)S(O)₂R″, C₁-C₃ alkyl, C₁-C₃ hydroxyalkyl, C₁-C₃ haloalkyl, C₂-C₄ alkenyl, (CH₂)ₒX, —SR″, and aryl;
o is 0 or 1;
Each R^{a} is independently selected from the group consisting of C₁-C₆ alkyl, C₁-C₃ alkoxy, —C(O)R″, and aralkyl;
J is selected from

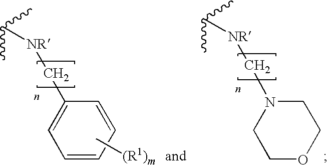

m is or 0 or 1;
n is 0, 1, or 2;
R¹ is halo, —CN, —NH₂, C₁-C₃ alkoxy, aryloxy, —C(O)N(H)R′, —C(O)OR″, heteroaryl optionally substituted with at least one C₁-C₃ alkyl, or —(CH₂)_{q}X;
q is 0 or 1;

### D is:

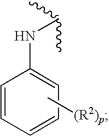

R² is selected from the group consisting of —O(CH₂)ₒNR′R″, —N(H)C(O)O(CH₂)ₒNR′R″, —(CH₂)ₒX, and —CH₂S(O)₂X;
p is 1;
o is 1 or 2;
R′ is —H, C₁-C₄ alkyl;
R″ is C₁-C₄ alkyl; and
X is heterocyclyl or heteroaryl.

It is to be understood that reference to compounds of formula (I) above, following herein, refers to compounds within the scope of formula (I) as defined above with respect to A, D, J, R, R^{a}, R¹, R², R′, R″, and X unless specifically limited otherwise.

It is understood that substituent bonding locations having an unfilled valence are indicated by

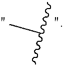

The appropriate attachments are further illustrated in the working examples recited below.

A is selected from —H, aryl optionally substituted with at least one R group, and heteroaryl optionally substituted with at least one R^{a} group, where R and R^{a} are as defined elsewhere herein. In one embodiment, A is aryl substituted with at least one R group. In certain embodiments, A is aryl substituted with one R group. In alternate embodiments, A is aryl substituted with two R groups. In additional embodiments, A is aryl substituted with three R groups. In another embodiment, A is heteroaryl substituted with at least one R^{a} group. In particular embodiments, the heteroaryl is a C₂-C₉ heteroaryl. In certain embodiments, A is heteroaryl substituted with one R^{a} group. In a particular embodiment, A is heteroaryl. In certain embodiments, A is selected from furanyl, 1H-indazolyl, pyridinyl, pyrimidinyl, thiophenyl, benzodioxolyl, thianthrenyl, benzofuranyl, and quinolinyl.

Each R is independently selected from the group consisting of halo, —OH, —NH₂, —CN, C₁-C₃ alkoxy, aryloxy, aralkoxy, —C(O)R″, —C(O)OR″, —C(O)OH, —C(O)H, —C(O)NR′R″, —NO₂, —N(H)C(O)R″, —N(H)S(O)₂R″, C₁-C₃ alkyl, C₁-C₃ hydroxyalkyl, C₁-C₃ haloalkyl, C₂-C₄ alkenyl, (CH₂)ₒX, —SR″, and aryl. In certain embodiments, at least one R is C₁-C₃ alkoxy. In particular embodiments, at least one R is methoxy or ethoxy. In alternate embodiments, at least one R is halo or haloalkyl. In particular embodiments, at least one R is fluoro. In other embodiments, at least one R is chloro. In certain embodiments, at least one R is —C(O)R″, —CHO, —C(O)NR′R″, or —C(O)OH. In alternate embodiments, at least one R is —NH. In further embodiments, at least one R is R is —CN. In other embodiments, at least one R is C₁-C₃ alkyl or C₂-C₄ alkenyl.

Each R^{a} is independently selected from the group consisting of C₁-C₆ alkyl, C₁-C₃ alkoxy, —C(O)R″, and aralkyl. In some embodiments, at least one R^{a} is C₁-C₆ alkyl such as, for example, methyl, ethyl, propyl, isopropyl, butyl, or isobutyl. In other embodiments, at least one R^{a} is C₁-C₃ alkoxy such as, for example, methoxy or ethoxy. In alternate embodiments, at least one R^{a} is aralkyl. In particular embodiments, R^{a} is benzyl.

J is selected from

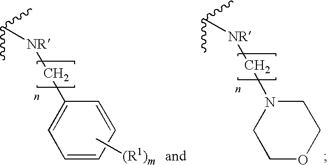

In particular embodiments, J is:

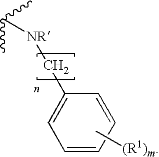

Where m is 1, R¹ is selected from halo, —CN, —NH₂, C₁-C₃ alkoxy, aryloxy, —C(O)N(H)R′, —C(O)OR″, heteroaryl optionally substituted with at least one C₁-C₃ alkyl, and —(CH₂)_{q}X. In one embodiment, R¹ is C₁-C₃ alkoxy. In particular embodiments, R¹ is methoxy. In other embodiments, R¹ is —C(O)N(H)R′. In further embodiments, R¹ is halo. In particular embodiments, R¹ is fluoro.

D is:

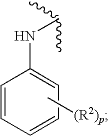

R² is selected from the group consisting of —O(CH₂)ₒNR′R″, —N(H)C(O)O(CH₂)ₒNR′R″, —(CH₂)ₒX, and —CH₂S(O)₂X. In particular embodiments, R² is —O(CH₂)ₒNR′R″. In other embodiments, R² is —N(H)C(O)O(CH₂)ₒNR′R″. In further embodiments, R² is selected from —(CH₂)ₒX, and —CH₂S(O)₂X. In certain embodiments, R² is —O(CH₂)₂N(CH₂CH₃)₂.

R′ is —H or C₁-C₄ alkyl. In some embodiments, R′ is —H. In other embodiments, R′ is C₁-C₄ alkyl. In particular embodiments, R′ is methyl. In alternate embodiments, R′ is ethyl. In additional embodiments, R′ is selected from n-propyl, isopropyl, n-butyl, isobutyl, and t-butyl.

R″ is C₁-C₄ alkyl. In particular embodiments, R″ is methyl. In alternate embodiments, R″ is ethyl. In additional embodiments, R″ is selected from n-propyl, isopropyl, n-butyl, isobutyl, and t-butyl.

X is heterocyclyl or heteroaryl. In some embodiments, X is heterocyclyl. In certain embodiments, X is a 5-, 6-, 7-, 8-, or 9-membered heterocyclyl. In particular embodiments, X is morpholinyl. In other embodiments, X is heteroaryl. In certain embodiments, X is C₂-C₉ heteroaryl. In particular embodiments, X is triazolyl.

It is to be understood that the present invention covers all combinations of groups in the embodiments described hereinabove.

Specific examples of compounds of the present invention include the following:
N²-(4-{[2-(diethylamino)ethyl]oxy}phenyl)-N⁴-[2-(methyloxy)phenyl]-5-(1H-pyrazol-4-yl)-2,4-pyrimidinediamine;
N²-(4-{[2-(diethylamino)ethyl]oxy}phenyl)-5-(1H-indazol-5-yl)-N⁴-[2-(methyloxy)phenyl]-2,4-pyrimidinediamine;
[4-(2-[(4-{[2-(diethylamino)ethyl]oxy}phenyl)amino]-4-{[2-(methyloxy)phenyl]amino}-5-pyrimidinyl)phenyl]methanol;
N²-(4-{[2-(diethylamino)ethyl]oxy}phenyl)-N⁴-[2-(methyloxy)phenyl]-5-[5-(methyloxy)-3-pyridinyl]-2,4-pyrimidinediamine;
4-(2-[(4-{[2-(diethylamino)ethyl]oxy}phenyl)amino]-4-{[2-(methyloxy)phenyl]amino}-5-pyrimidinyl)phenol;
N²-(4-{[2-(diethylamino)ethyl]oxy}phenyl)-N⁴-[2-(methyloxy)phenyl]-5,5′-bipyrimidine-2,4-diamine;
N²-(4-{[2-(diethylamino)ethyl]oxy}phenyl)-N⁴-[2-(methyloxy)phenyl]-5-(3-pyridinyl)-2,4-pyrimidinediamine
N²-(4-{[2-(diethylamino)ethyl]oxy}phenyl)-N⁴-[2-(methyloxy)phenyl]-5-(3-quinolinyl)-2,4-pyrimidinediamine;
5-(2-chlorophenyl)-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[1-(phenylmethyl)-1H-pyrazol-4-yl]-2,4-pyrimidinediamine;
N²-(4-{[2-(diethylamino)ethyl]oxy}phenyl)-N⁴-[2-(methyloxy)phenyl]-5-(3-quinolinyl)-2,4-pyrimidinediamine;
N²-(4-{[2-(diethylamino)ethyl]oxy}phenyl)-5-(3,5-dimethyl-1H-pyrazol-4-yl)-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)phenol;
N²-(4-{[2-(diethylamino)ethyl]oxy}phenyl)-N⁴,5-bis[2-(methyloxy)phenyl]-2,4-pyrimidinediamine;
N²-(4-{[2-(diethylamino)ethyl]oxy}phenyl)-5-(3-furanyl)-N⁴-[2-(methyloxy)phenyl]-2,4-pyrimidinediamine;
1-[5-(2-[(4-{[2-(diethylamino)ethyl]oxy}phenyl)amino]-4-{[2-(methyloxy)phenyl]amino}-5-pyrimidinyl)-2-thienyl]ethanone;
2-(2-[(4-{[2-(diethylamino)ethyl]oxy}phenyl)amino]-4-{[2-(methyloxy)phenyl]amino}-5-pyrimidinyl)phenol;
N²-(4-{[2-(diethylamino)ethyl]oxy}phenyl)-5-(3-fluorophenyl)-N⁴-[2-(methyloxy)phenyl]-2,4-pyrimidinediamine;
[3-(2-[(4-{[2-(diethylamino)ethyl]oxy}phenyl)amino]-4-{[2-(methyloxy)phenyl]amino}-5-pyrimidinyl)phenyl]methanol;
5-(1,3-benzodioxol-5-yl)-N²-(4-{[2-(diethylamino)ethyl]oxy}phenyl)-N⁴-[2-(methyloxy)phenyl]-2,4-pyrimidinediamine;
5-(1-benzothien-3-yl)-N²-(4-{[2-(diethylamino)ethyl]oxy}phenyl)-N⁴-[2-(methyloxy)phenyl]-2,4-pyrimidinediamine;
N⁴-[2-(methyloxy)phenyl]-N²-{3-[2-(4-morpholinyl)ethyl]phenyl}-5,5′-bipyrimidine-2,4-diamine;
N-(1-methylpropyl)-2-[(5-(1H-pyrazol-4-yl)-2-{[4-(1H-1,2,4-triazol-1-ylmethyl)phenyl]amino}-4-pyrimidinyl)amino]benzamide;
N⁴-[2-(3-fluorophenypethyl]-N²-[4-(1H-1,2,4-triazol-1-ylmethyl)phenyl]-5,5′-bipyrimidine-2,4-diamine;
N²-(4-{[2-(diethylamino)ethyl]oxy}phenyl)-5-(3,4-difluorophenyl)-N⁴-[2-(methyloxy)phenyl]-2,4-pyrimidinediamine;
N²-(4-{[2-(diethylamino)ethyl]oxy}phenyl)-5-(2-fluorophenyl)-N⁴-[2-(methyloxy)phenyl]-2,4-pyrimidinediamine;
1-[4-(2-[(4-{[2-(diethylamino)ethyl]oxy}phenyl)amino]-4-{[2-(methyloxy)phenyl]amino}-5-pyrimidinyl)phenyl]ethanone;
N²-(4-{[2-(diethylamino)ethyl]oxy}phenyl)-N⁴-[2-(methyloxy)phenyl]-5-phenyl-2,4-pyrimidinediamine;
4-(2-[(4-{[2-(diethylamino)ethyl]oxy}phenyl)amino]-4-{[2-(methyloxy)phenyl]amino}-5-pyrimidinyl)benzoic acid;
4-(2-[(4-{[2-(diethylamino)ethyl]oxy}phenyl)amino]-4-{[2-(methyloxy)phenyl]amino}-5-pyrimidinyl)benzoic acid;
N⁴-[2-(methyloxy)phenyl]-N²-[4-(1H-1,2,4-triazol-1-ylmethyl)phenyl]-5,5′-bipyrimidine-2,4-diamine;
N²-(4-{[2-(diethylamino)ethyl]oxy}phenyl)-N⁴,5-bis[2-(methyloxy)phenyl]-2,4-pyrimidinediamine
5-(3-aminophenyl)-N²-(4-{[2-(diethylamino)ethyl]oxy}phenyl)-N⁴-[2-(methyloxy)phenyl]-2,4-pyrimidinediamine;
4-(2-[(4-{[2-(diethylamino)ethyl]oxy}phenyl)amino]-4-{[2-(methyloxy)phenyl]amino}-5-pyrimidinyl)benzaldehyde;
N²-(4-{[2-(diethylamino)ethyl]oxy}phenyl)-N⁴-[2-(methyloxy)phenyl]-5-(2-methylphenyl)-2,4-pyrimidinediamine;
5-(3,4-dichlorophenyl)-N²-(4-{[2-(diethylamino)ethyl]oxy}phenyl)-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)benzonitrile;
N²-(4-{[2-(diethylamino)ethyl]oxy}phenyl)-N⁴-[2-(methyloxy)phenyl]-5-[3-(methyloxy)phenyl]-2,4-pyrimidinediamine;
N²-(4-{[2-(diethylamino)ethyl]oxy}phenyl)-N⁴-[2-(methyloxy)phenyl]-5-[4-(methyloxy)phenyl]-2,4-pyrimidinediamine;
N-(1-methylpropyl)-2-[(2-{[4-(1H-1,2,4-triazol-1-ylmethyl)phenyl]amino}-5,5′-bipyrimidin-4-yl)amino]benzamide;
2-(diethylamino)ethyl {4-[(4-{[2-(methyloxy)phenyl]amino}-5,5′-bipyrimidin-2-yl)amino]phenyl}carbamate;
3-({2-[(4-{[2-(diethylamino)ethyl]oxy}phenyl)amino]-5,5′-bipyrimidin-4-yl}amino)benzonitrile;
N²-(4-{[2-(diethylamino)ethyl]oxy}phenyl)-N⁴-[2-(methyloxy)phenyl]-5-(3-methylphenyl)-2,4-pyrimidinediamine;
N²-(4-{[2-(diethylamino)ethyl]oxy}phenyl)-2′,4′-bis(methyloxy)-N⁴-[2-(methyloxy)phenyl]-5,5′-bipyrimidine-2,4-diamine;
N²-(4-{[2-(diethylamino)ethyl]oxy}phenyl)-N⁴-[2-(methyloxy)phenyl]-5-[1-(2-methylpropyl)-1H-pyrazol-4-yl]-2,4-pyrimidinediamine;
N²-(4-{[2-(diethylamino)ethyl]oxy}phenyl)-N⁴-[2-(methyloxy)phenyl]-5-[2-(methylthio)phenyl]-2,4-pyrimidinediamine;
N-[4-(2-[(4-{[2-(diethylamino)ethyl]oxy}phenyl)amino]-4-{[2-(methyloxy)phenyl]amino}-5-pyrimidinyl)phenyl]acetamide;
5-[2,4-bis(methyloxy)phenyl]-N²-(4-{[2-(diethylamino)ethyl]oxy}phenyl)-N⁴-[2-(methyloxy)phenyl]-2,4-pyrimidinediamine;
N⁴-[3-(2-methyl-1,3-thiazol-5-yl)phenyl]-N²-[4-(1H-1,2,4-triazol-1-ylmethyl)phenyl]-5,5′-bipyrimidine-2,4-diamine;
N⁴-[3-(2-methyl-1,3-thiazol-5-yl)phenyl]-N²-{3-[2-(4-morpholinyl)ethyl]phenyl}-5,5′-bipyrimidine-2,4-diamine;
N²-(4-{[2-(diethylamino)ethyl]oxy}phenyl)-5-(4-ethenylphenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(4-methylphenyl)-2,4-pyrimidinediamine;
N²-(4-{[2-(diethylamino)ethyl]oxy}phenyl)-5-[1-(3-methylbutyl)-1H-pyrazol-4-yl]-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(1H-pyrrolo[2,3-b]pyridin-4-yl)-2,4-pyrimidinediamine;
5-(3-chloro-4-fluorophenyl)-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(8-quinolinyl)-2,4-pyrimidinediamine;
N²-(4-{[2-(diethylamino)ethyl]oxy}phenyl)-5-(4-ethylphenyl)-N⁴-[2-(methyloxy)phenyl]-2,4-pyrimidinediamine;
3-[(2-{[4-(1H-1,2,4-triazol-1-ylmethyl)phenyl]amino}-5,5′-bipyrimidin-4-yl)amino]benzonitrile;
N²-(4-{[2-(diethylamino)ethyl]oxy}phenyl)-N⁴-[2-(methyloxy)phenyl]-5-(2-naphthalenyl)-2,4-pyrimidinediamine;
N²-(4-{[2-(diethylamino)ethyl]oxy}phenyl)-5-[3,5-dimethyl-4-(methyloxy)phenyl]-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)benzamide;
5-[3,4-bis(methyloxy)phenyl]-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-5-(2-fluoro-4-biphenylyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[4-(methylthio)phenyl]-2,4-pyrimidinediamine;
5-[5-chloro-2-(methyloxy)phenyl]-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[3-(trifluoromethyl)phenyl]-2,4-pyrimidinediamine;
N²-(4-{[2-(diethylamino)ethyl]oxy}phenyl)-N⁴-[2-(methyloxy)phenyl]-5-(5-quinolinyl)-2,4-pyrimidinediamine;
5-[2,5-bis(methyloxy)phenyl]-N²-(4-{[2-(diethylamino)ethyl]oxy}phenyl)-N⁴-[2-(methyloxy)phenyl]-2,4-pyrimidinediamine;
1-[3-(2-[(4-{[2-(diethylamino)ethyl]oxy}phenyl)amino]-4-{[2-(methyloxy)phenyl]amino}-5-pyrimidinyl)phenyl]ethanone;
N²-(4-{[2-(diethylamino)ethyl]oxy}phenyl)-5-{3-fluoro-4-[(phenylmethyl)oxy]phenyl}-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(6-quinolinyl)-2,4-pyrimidinediamine;
N²-(4-{[2-(diethylamino)ethyl]oxy}phenyl)-N⁴-{[2-(methyloxy)phenyl]methyl}-5-(1H-pyrazol-4-yl)-2,4-pyrimidinediamine;
N²-(4-{[2-(diethylamino)ethyl]oxy}phenyl)-N⁴-[3-(1-piperidinylmethyl)phenyl]-5,5′-bipyrimidine-2,4-diamine;
N²-(4-{[2-(diethylamino)ethyl]oxy}phenyl)-5-[3-(ethyloxy)phenyl]-N⁴-[2-(methyloxy)phenyl]-2,4-pyrimidinediamine;
N²-(4-{[2-(diethylamino)ethyl]oxy}phenyl)-5-[4-(ethyloxy)phenyl]-N⁴-[2-(methyloxy)phenyl]-2,4-pyrimidinediamine;
N²-(4-{[2-(diethylamino)ethyl]oxy}phenyl)-N⁴-[2-(3-fluorophenyl)ethyl]-5,5′-bipyrimidine-2,4-diamine;
N²-(4-{[2-(diethylamino)ethyl]oxy}phenyl)-N⁴-{[2-(methyloxy)phenyl]methyl}-5,5′-bipyrimidine-2,4-diamine;
3-(2-[(4-{[2-(diethylamino)ethyl]oxy}phenyl)amino]-4-{[2-(methyloxy)phenyl]amino}-5-pyrimidinyl)benzoic acid;
N²-(4-{[2-(diethylamino)ethyl]oxy}phenyl)-N⁴-[2-(methyloxy)phenyl]-5-(1-thianthrenyl)-2,4-pyrimidinediamine;
N²-(4-{[2-(diethylamino)ethyl]oxy}phenyl)-N⁴-[2-(methyloxy)phenyl]-5-[4-(trifluoromethyl)phenyl]-2,4-pyrimidinediamine;
5-(1-benzofuran-2-yl)-N²-(4-{[2-(diethylamino)ethyl]oxy}phenyl)-N⁴-[2-(methyloxy)phenyl]-2,4-pyrimidinediamine;
N²-{3-[2-(4-morpholinyl)ethyl]phenyl}-N⁴-[2-(phenyloxy)phenyl]-2,4-pyrimidinediamine;
N²-(4-{[2-(diethylamino)ethyl]oxy}phenyl)-5-[2-(ethyloxy)phenyl]-N⁴-[2-(methyloxy)phenyl]-2,4-pyrimidinediamine;
N⁴-[2-(3-fluorophenypethyl]-5-(1H-pyrazol-4-yl)-N²-[4-(1H-1,2,4-triazol-1-ylmethyl)phenyl]-2,4-pyrimidinediamine;
N²-(4-{[2-(diethylamino)ethyl]oxy}phenyl)-N⁴-[2-(methyloxy)phenyl]-5-(4-propylphenyl)-2,4-pyrimidinediamine;
N⁴-[(2-aminophenyl)methyl]-N²-(4-{[2-(diethylamino)ethyl]oxy}phenyl)-5,5′-bipyrimidine-2,4-diamine;
N⁴-{[2-(methyloxy)phenyl]methyl}-N²-{3-[2-(4-morpholinyl)ethyl]phenyl}-5,5′-bipyrimidine-2,4-diamine;
5-(2-biphenylyl)-N²-(4-{[2-(diethylamino)ethyl]oxy}phenyl)-N⁴-[2-(methyloxy)phenyl]-2,4-pyrimidinediamine;
5-(2-biphenylyl)-N²-(4-{[2-(diethylamino)ethyl]oxy}phenyl)-N⁴-[2-(methyloxy)phenyl]-2,4-pyrimidinediamine;
N⁴-[2-(3-fluorophenypethyl]-N²-(4-{[(4-methyl-1-piperazinyl)sulfonyl]methyl}phenyl)-5-(1H-pyrazol-4-yl)-2,4-pyrimidinediamine;
5-(3-biphenylyl)-N²-(4-{[2-(diethylamino)ethyl]oxy}phenyl)-N⁴-[2-(methyloxy)phenyl]-2,4-pyrimidinediamine;
N⁴-{[2-(methyloxy)phenyl]methyl}-N²-[4-(1H-1,2,4-triazol-1-ylmethyl)phenyl]-5,5′-bipyrimidine-2,4-diamine;
4-(2-[(4-{[2-(diethylamino)ethyl]oxy}phenyl)amino]-4-{[2-(methyloxy)phenyl]amino}-5-pyrimidinyl)benzonitrile;
methyl 4-(2-[(4-{[2-(diethylamino)ethyl]oxy}phenyl)amino]-4-{[2-(methyloxy)phenyl]amino}-5-pyrimidinyl)benzoate;
methyl 4-(2-[(4-{[2-(diethylamino)ethyl]oxy}phenyl)amino]-4-{[2-(methyloxy)phenyl]amino}-5-pyrimidinyl)benzoate;
5-[3,5-bis(trifluoromethyl)phenyl]-N²-(4-{[2-(diethylamino)ethyl]oxy}phenyl)-N⁴-[2-(methyloxy)phenyl]-2,4-pyrimidinediamine; and
N²-(3-{[2-(dimethylamino)ethyl]oxy}phenyl)-N⁴-[2-(methyloxy)phenyl]-5-(1H-pyrazol-4-yl)-2,4-pyrimidinediamine hydrochloride.

Salts of formula (I) are also encompassed. Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula (I), as well as salts and solvates thereof, may be administered as the raw chemical, it is possible to present the active ingredient as a pharmaceutical composition. Accordingly, the invention further provides pharmaceutical compositions, which include therapeutically effective amounts of compounds of the formula (I) and salts and solvates thereof, and one or more pharmaceutically acceptable carriers, diluents, or excipients. The compounds of the formula (I) and salts and solvates thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts and solvates thereof, with one or more pharmaceutically acceptable carriers, diluents or excipients.

Pharmaceutical formulations may be presented in unit dose forms containing a predetermined amount of active ingredient per unit dose. Such dosage may vary depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle.

Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release, as for example, by coating or embedding particulate material in polymers, wax or the like.

The compounds of formula (I), and salts and solvates thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. The compounds of formula (I) and salts and solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the human or other animal, the precise condition requiring treatment and its severity, the nature of the formulation, and the route of administration, and will ultimately be at the discretion of the attendant physician or veterinarian. An effective amount of a salt or solvate thereof, may be determined as a proportion of the effective amount of the compound of formula (I) per se. It is envisaged that similar dosages would be appropriate for treatment of the other conditions referred to above.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I).

Compounds of general formula (I) can be prepared according to the synthetic sequences illustrated in Scheme 1 and further detailed in the Examples section following.

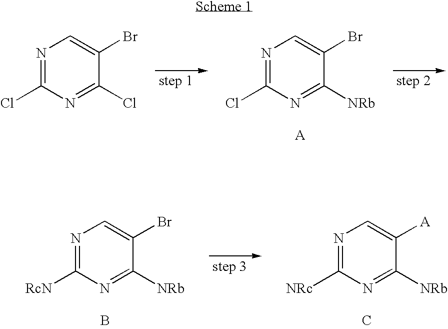

Selective 4-chloro displacement of 5-bromo-2,4-dichloropyrimidine can be achieved to give A in the presence of aniline and an amine base in an appropriate solvent such as isopropyl alcohol or 2-propanol. 4-Anilino-pyrimidine A can be converted to the dianilino compound B by treatment with aniline in the presence of and acid, either concentrated HCL or 3N HCl, in an appropriate solvent such as isopropyl alcohol or 2-propanol. Compounds C can be made by reaction of boronate esters or boronic acids with B under Suzuki reaction conditions. The Suzuki reaction is well described in the synthetic chemistry literature, and is a method for preparing biaryl compounds from aryl halides and either boronate esters or boronic acids. The reaction may be performed in a variety of solvents or mixtures of solvents (including but not limited to DMF, EtOH, DME, toluene, dioxane, THF, water) in the presence of a catalyst (including but not limited to Pd(Ph₃P)₄ and Pd(Ph₃P)₂Cl₂) and a base (including but not limited to Et₃N, K₂CO₃, Na₂CO₃) at temperatures ranging from 80° C. to 180° C.

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megaHertz); mol (moles);
mmol (millimoles); rt (room temperature);
min (minutes); h (hours);
mp (melting point); TLC (thin layer chromatography);
Tᵣ (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); AcOEt (ethyl acetate);
DME (1,2-dimethoxyethane); DCM (dichloromethane);
DCE (dichloroethane); DMF (N,N-dimethylformamide);
DMPU (N,N′-dimethylpropyleneurea); CDI (1,1′-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid);
EDC (1-[(3-dimethylamino) propyl]-3-ethylcarbodiimide hydrochloride);
BOC (tert-butyloxycarbonyl); FMOC (9-fluorenylmethoxycarbonyl);
DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
ATP (adenosine triphosphate); HRP (horseradish peroxidase);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluroniumhexafluoro phosphate).
HEPES (4-(2-hydroxyethyl)-1-piperazine ethane sulfonic acid);
DPPA (diphenylphosphoryl azide);
fHNO₃ (fuming HNO₃); and
EDTA (ethylenediaminetetraacetic acid).

### Intermediate Example 1

### General Procedure for the Installation of Amines at the 4 Position

### Preparation of 5-bromo-2-chloro-N-[2-(methyloxy)phenyl]-4-pyrimidinamine

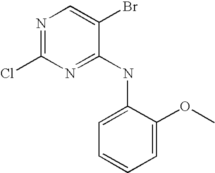

To solid 5-bromo-2,4-dichloropyrimidine (2.0 g, 1.0 eq) dissolved in n-butanol (0.4M) was added 2-(methyloxy)aniline (0.99 mL, 1.0 eq) and diisopropylethylamine (2.3 mL, 1.5 eq). The solution was heated at 110° C. for ca. 5H. Add 50 mL cold water and allow the mixture to cool to ambient temperature. Filter white solids and wash with diethyl ether (2×10 mL) to give 5-bromo-2-chloro-N-[2-(methyloxy)phenyl]-4-pyrimidinamine in 75% yield.

1H NMR (400 MHz, DMSO-D6) ppm 2.5 (dt, J=3.5, 1.7 Hz, 10H) 3.3 (s, 15H) 3.8 (s, 3H) 7.0 (td, J=7.6, 1.3 Hz, 1H) 7.1 (dd, J=8.3, 1.4 Hz, 1H) 7.2 (m, 1H) 7.7 (dd, J=8.0, 1.6 Hz, 1H) 8.7 (s, 1H). ¹³C NMR (400 MHz, DMSO-D6) ppm 157.9, 157.8, 157.7, 151.8, 126.4, 126.1, 124.2, 120.4, 111.8, 103.4, 55.9. LC/MS: m/z 318 (M+1)⁺.

### Intermediate Example 2

### General Procedure for Installation of Anilines at the 2 Position

### Preparation of 5-bromo-N²-(4-{[2-(diethylamino)ethyl]oxy}phenyl)-N⁴-[2-(methyloxy)phenyl]-2,4-pyrimidinediamine

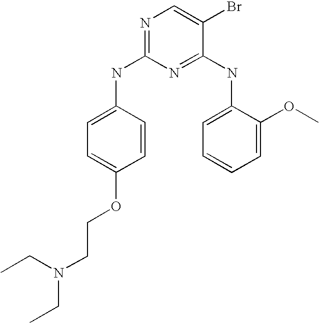

To solid 5-bromo-2-chloro-N-[2-(methyloxy)phenyl]-4-pyrimidinamine (1.0 g, 1.0 eq) dissolved in n-butanol (0.4M) was added 4-{[2-(diethylamino)ethyl]oxy}aniline hydrochloride (780 mgs, 1.0 eq) and 3N HCl (1 mL). After heating at 110° C. for 5 hours pour hot reaction mixture into cold water and filter. Collect filtrate, remove solvents in vacuo and dissolve remaining residue in ethyl acetate. Wash (2×) with saturated NaHCO₃ and brine. Dry over magnesium sulfate, filter and remove solvents in vacuo leaving 5-bromo-N²-(4-{[2-(diethylamino)ethyl]oxy}phenyl)-N⁴-[2-(methyloxy)phenyl]-2,4-pyrimidinediamine as a pale brown solid in 65% yield.

1H NMR (400 MHz, DMSO-D6) δ ppm 1.0 (t, J=7.1 Hz, 4H) 2.5 (dt, J=3.7, 1.8 Hz, 12H) 2.5 (t, J=7.0 Hz, 3H) 2.7 (t, J=6.3 Hz, 2H) 3.3 (s, 4H) 3.8 (s, 2H) 3.9 (t, J=6.3 Hz, 1H) 6.8 (d, J=9.0 Hz, 1H) 6.9 (ddd, J=8.2, 6.0, 2.5 Hz, 1H) 7.1 (m, 2H) 7.4 (d, J=8.8 Hz, 1H) 8.1 (m, 1H). LC/MS: m/z 245 (M+1)⁺.

### Example 3

### General Suzuki Coupling Procedure for the Installation of Aryl Group at the 5 Position

### N²-(4-{[2-(diethylamino)ethyl]oxy}phenyl)-N⁴-[2-(methyloxy)phenyl]-5-(1H-pyrazol-4-yl)-2,4-pyrimidinediamine

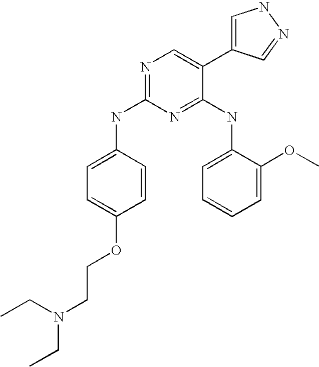

To a 10 mL microwave vial equipped with a magnetic stir bar add 5-bromo-N²-(4-{[2-(diethylamino)ethyl]oxy}phenyl)-N⁴-[2-(methyloxy)phenyl]-2,4-pyrimidine diamine (48.6 mgs, 1.0 eq), 1-tert-butoxycarbonyl-4-1H-pyrazolboronic acid, pinacol ester (44.1 mgs, 1.5 eq), and PdCl₂(PPh₃)₂ (7 mgs, 0.01 eq), in dimethylformamide (3 mLs) and 2N Na₂CO₃ (1 mL). Heat the reaction mixture in an Emrys microwave at 160° C. for 10 minutes. Once cooled to ambient temperature and filter mixture through pad of celite. Gravity filter organics through an SCX ion exchange column (previously washed with methanol) and wash resin with dichloromethane (3×). Wash resin with 2N NH₃/MeOH (3×3 mLs) and collect filtrate. Remove solvents in vacuo and purify on Agilent preparatory liquid chromatograph system. (10 to 100% acetonitrile/0.02% aqueous NH₄OH over 14 min)

1H NMR (400 MHz, DMSO-D6) δ ppm 0.9 (t, J=7.1 Hz, 6H) 2.5 (q, J=7.4 Hz, 4H) 2.7 (t, J=5.9 Hz, 2H) 3.8 (s, 3H) 3.9 (t, J=6.1 Hz, 2H) 6.8 (d, J=8.1 Hz, 2H) 6.9 (m, 1H) 7.0 (s, 3H) 7.5 (d, J=8.6 Hz, 2H) 7.8 (s, 2H) 8.0 (s, 1H) 8.5 (d, J=8.8 Hz, 1H) 9.1 (s, 1H) 7.94 (brs, 1H). LC/MS: m/z 474 (M+1)⁺.

### Example 4

### N²-(4-[{2-(diethylamino)ethyl]oxy}phenyl)-5-(1H-indazol-5-yl)-N⁴-[2-(methyloxy)phenyl]-2,4-pyrimidinediamine

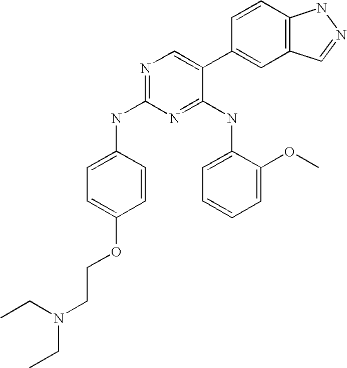

The title compound was prepared by the general procedure in Example 3. 1H NMR 1H 1H NMR (400 MHz, DMSO-D6) δ ppm 0.9 (t, J=7.1 Hz, 6H) 2.5 (q, J=7.1 Hz, 4H) 2.7 (t, J=6.1 Hz, 2H) 3.6 (s, 3H) 4.0 (t, J=6.6 Hz, 2H) 6.8 (d, J=8.8 Hz, 2H) 6.9 (m, 1H) 7.0 (m, 2H) 7.4 (dd, J=8.5, 1.6 Hz, 1H) 7.6 (d, J=9.0 Hz, 2H) 7.7 (d, J=8.4 Hz, 1H) 7.7 (s, 1H) 7.9 (s, 1H) 8.0 (s, 1H) 8.1 (s, 1H) 8.4 (m, 2H) 9.1 (s, 1H) 13.2 (s, 1H). LC/MS: m/z 524 (M+1)⁺.

### Example 5

### [4-(2-[(4-{[2-(diethylamino)ethyl]oxy}phenyl)amino]-4-{[2-(methyloxy)phenyl]amino}-5-pyrimidinyl)phenyl]methanol

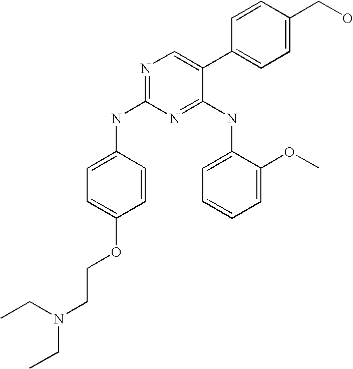

The title compound was prepared by the general procedure in Example 3. 1H NMR (400 MHz, METHANOL-D4) δ ppm 1.1 (t, J=7.1 Hz, 6H) 2.7 (q, J=7.4 Hz, 4H) 3.0 (t, J=5.2 Hz, 2H) 3.7 (s, 3H) 4.1 (t, J=5.7 Hz, 2H) 4.7 (s, 2H) 6.8 (t, J=8.4 Hz, 1H) 6.9 (m, 4H) 7.5 (dd, J=8.4, 4.4 Hz, 4H) 7.5 (m, 2H) 7.8 (s, 1H) 8.4 (d, J=8.1 Hz, 1H). LC/MS: 514 m/z (M+1)⁺.

### Example 6

### N²-(4-{[2-(diethylamino)ethyl]oxy}phenyl)-N⁴-[2-(methyloxy)phenyl]-5-[5-(methyloxy)-3-pyridinyl]-2,4-pyrimidinediamine

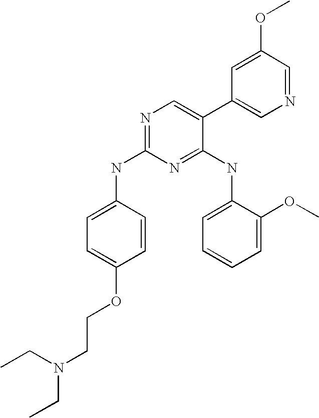

The title compound was prepared by the general procedure in Example 3. 1H NMR (300 MHz, METHANOL-D4) δ ppm 1.2 (t, J=7.2 Hz, 6H) 2.8 (q, J=7.2 Hz, 4H) 3.0 (t, J=5.8 Hz, 2H) 3.8 (s, 3H) 4.0 (s, 3H) 4.1 (t, J=5.7 Hz, 2H) 6.9 (m, 3H) 7.0 (m, 2H) 7.5 (m, 2H) 7.6 (dd, J=2.7, 1.8 Hz, 1H) 8.0 (s, 1H) 8.3 (d, J=1.7 Hz, 1H) 8.3 (d, J=2.8 Hz, 1H). LC/MS: m/z 513 (M−1).

### Example 7

### 4-(2-[4-{[2-(diethylamino)ethyl]oxy}phenyl)amino]-4-{[2-(methyloxy)phenyl]amino}-5-pyrimidinyl)phenol

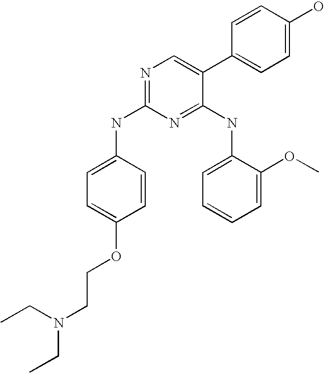

The title compound was prepared by the general procedure in Example 4 with the addition of Et₃N as a base. 1H NMR (400 MHz, DMSO-d6) ppm 2.37 (s, 3H), 3.72 (s, 3H), 3.78 (s, 3H), 4.49 (m, 2H), 6.86 (m, 2H), 7.33-7.40 (m, 5H), 7.56 (m, 1H), 7.60 (m, 1H), 7.70 (m, 2H), 7.74 (m, 2H), 9.01 (brs, 1H), 9.19 (brs, 1H), 11.70 (brs, 1H). LC/MS: m/z 494 (M+1)⁺.

### Example 8

### N²-(4-{[2-(diethylamino)ethyl]oxy}phenyl)-N⁴-[2-(methyloxy)phenyl]-5,5′-bipyrimidine-2,4-diamine

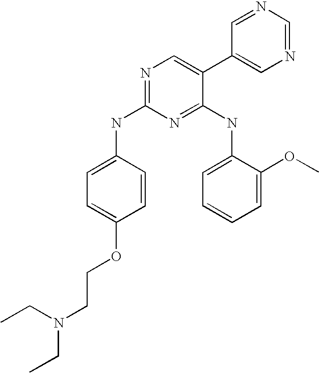

The title compound was prepared by the general procedure in Example 4 with the addition of Et₃N as a base. 1H NMR (400 MHz, METHANOL-D4) δ ppm 1.1 (m, 6H) 2.7 (s, 4H) 3.0 (s, 2H) 3.8 (s, 4H) 4.1 (s, 2H) 6.9 (s, 3H) 7.0 (s, 1H) 7.1 (s, 1H) 7.4 (s, 2H) 7.9 (s, 1H) 8.0 (s, 1H) 8.9 (s, 2H) 9.1 (s, 1H) LC/MS: m/z 486 (M+1)⁺.

### Example 9

### N²-(4-{[2-(diethylamino)ethyl]oxy}phenyl)-N⁴-[2-(methyloxy)phenyl]-5-(3-pyridinyl)-2,4-pyrimidinediamine

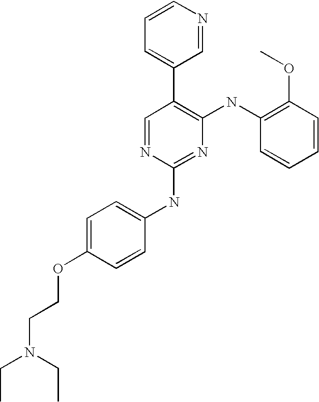

The title compound was prepared by the general procedure in Example 3. 1H NMR (300 MHz, METHANOL-D4) δ ppm 1.2 (t, J=7.2 Hz, 10H) 2.8 (d, J=7.2 Hz, 6H) 3.0 (s, 3H) 3.8 (s, 3H) 4.2 (s, 3H) 6.9 (s, 4H) 7.0 (s, 1H) 7.1 (s, 1H) 7.5 (s, 3H) 7.6 (s, 1H) 8.0 (s, 1H) 8.0 (s, 1H) 8.3 (s, 1H) 8.6 (s, 1H) 8.7 (s, 1H) LC/MS: m/z 485 (M+1)⁺.

### Example 10

### N²-(4-{[2-(diethylamino)ethyl]oxy}phenyl)-N⁴-[2-(methyloxy)phenyl]-5-(3-quinolinyl)-2,4-pyrimidinediamine

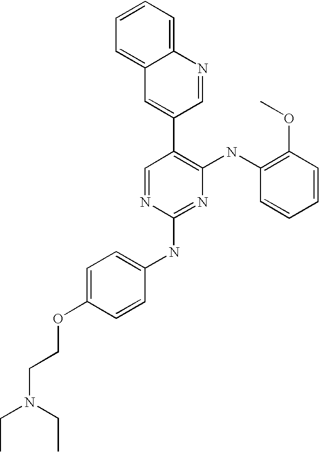

The title compound was prepared by the general procedure in Example 3. 1H NMR (400 MHz, DMSO-D6) δ ppm 1.0 (t, J=7.1 Hz, 7H) 2.5 (t, J=7.0 Hz, 6H) 2.7 (s, 2H) 3.4 (s, 3H) 4.0 (s, 2H) 6.8 (m, 2H) 6.9 (m, 2H) 7.0 (m, 1H) 7.2 (s, 1H) 7.5 (s, 2H) 7.8 (m, 3H) 8.0 (s, 1H) 8.1 (s, 1H) 8.2 (m, 1H) 8.5 (s, 1H) 9.2 (s, 1H) 9.4 (s, 1H) LC/MS: m/z 535 (M+1)⁺.

### Example 11

### 5-(2-chlorophenyl)-N²-(4-{[2-(diethylamino)ethyl]oxy}phenyl)-N⁴-[2-(methyloxy)phenyl]-2,4-pyrimidinediamine

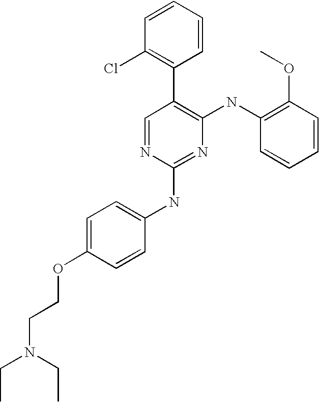

The title compound was prepared by the general procedure in Example 3. 1H NMR (300 MHz, METHANOL-D4) δ ppm 1.2 (t, J=7.2 Hz, 6H) 2.8 (q, J=7.2 Hz, 4H) 3.0 (t, J=5.7 Hz, 2H) 3.7 (s, 3H) 4.2 (t, J=5.7 Hz, 2H) 6.9 (m, 6H) 7.5 (m, 6H) 7.7 (m, 1H) 7.8 (s, 1H) 8.5 (dd, J=8.3, 1.1 Hz, 1H) LC/MS: m/z 518 (M+1)⁺.

### Example 12

### N²-(4-{[2-(diethylamino)ethyl]oxy}phenyl)-N⁴-[2-(methyloxy)phenyl]-5-[1-(phenylmethyl)-1H-pyrazol-4-yl]-2,4-pyrimidinediamine

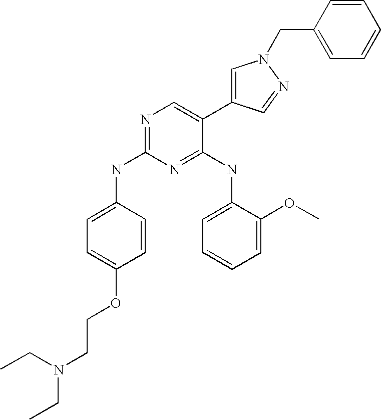

The title compound was prepared by the general procedure in Example 3. 1H NMR (400 MHz, DMSO-D6) δ ppm 1.0 (t, J=7.1 Hz, 6H) 2.5 (q, J=7.1 Hz, 5H) 2.7 (t, J=6.2 Hz, 2H) 3.7 (s, 3H) 4.0 (t, J=6.2 Hz, 2H) 5.4 (s, 2H) 6.8 (m, 2H) 6.9 (s, 1H) 7.0 (d, J=3.3 Hz, 2H) 7.3 (m, 3H) 7.4 (m, 2H) 7.5 (d, J=9.0 Hz, 2H) 7.7 (m, 2H) 8.0 (s, 1H) 8.2 (s, 1H) 8.5 (d, J=5.3 Hz, 1H) 9.1 (s, 1H) LC/MS: m/z 564 (M+1)⁺.

### Example 13

### N²-(4-{[2-(diethylamino)ethyl]oxy}phenyl)-N⁴-[2-(methyloxy)phenyl]-5-(3-quinolinyl)-2,4-pyrimidinediamine

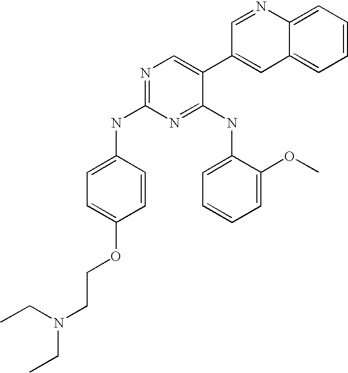

The title compound was prepared by the general procedure in Example 3. 1H NMR (400 MHz, METHANOL-D4) δ ppm 1.1 (t, J=7.1 Hz, 6H) 2.7 (d, J=7.1 Hz, 4H) 2.9 (s, 2H) 3.7 (s, 3H) 4.1 (s, 2H) 4.9 (s, 5H) 6.9 (m, 4H) 7.0 (m, 1H) 7.5 (m, 2H) 7.7 (ddd, J=8.1, 6.9, 1.1 Hz, 1H) 7.8 (ddd, J=8.6, 7.0, 1.5 Hz, 1H) 8.0 (m, 2H) 8.1 (dd, J=8.5, 0.8 Hz, 1H) 8.3 (d, J=7.7 Hz, 1H) 8.5 (d, J=2.2 Hz, 1H) 9.0 (d, J=2.2 Hz, 1H) LC/MS: m/z 535 (M+1)⁺.

### Example 14

### N²-(4-{[2-(diethylamino)ethyl]oxy}phenyl)-5-(3,5-dimethyl-1H-pyrazol-4-yl)-N⁴-[2-(methyloxy)phenyl]-2,4-pyrimidinediamine

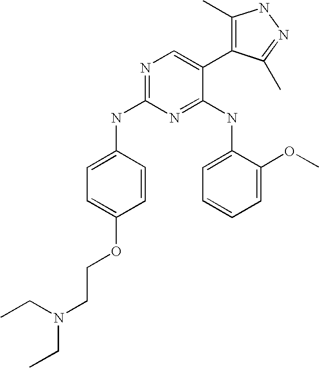

1H NMR (400 MHz, DMSO-D6) δ ppm 2.0 (s, 2H) 2.1 (d, J=13.4 Hz, 4H) 2.5 (q, J=7.1 Hz, 6H) 2.7 (t, J=6.2 Hz, 2H) 3.7 (s, 3H) 4.0 (t, J=6.2 Hz, 2H) 6.8 (m, 2H) 6.9 (m, 1H) 7.0 (m, 2H) 7.3 (s, 1H) 7.6 (d, J=9.1 Hz, 2H) 7.8 (s, 1H) 8.5 (dd, J=11.4, 7.0 Hz, 1H) 9.1 (s, 1H) 12.5 (s, 1H). LC/MS: m/z 500 (M−1).

The compounds in Table 1 were prepared essentially as described in Example 3 above.

TABLE 1

| Example | Structure | Name | LCMS |
|---|---|---|---|
| 15 | 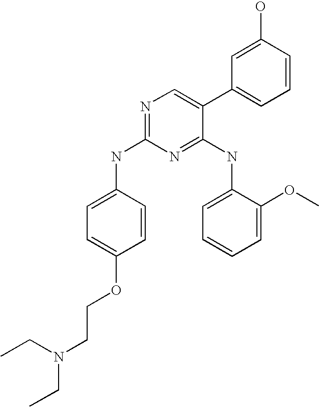 | 3-(2-[(4-{[2- (diethylamino)ethyl]oxy} phenyl)amino]-4-{[2- (methyloxy)phenyl]amino}- 5-pyrimidinyl)phenol | MS (ES+) m/e 500 [M + H]⁺ |
| 16 | 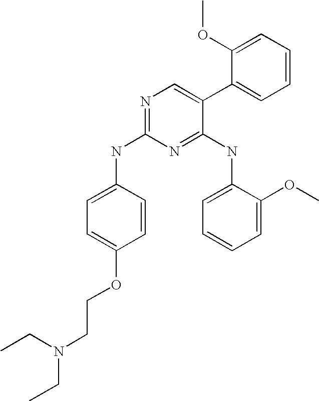 | N²-(4-{[2- (diethylamino)ethyl]oxy} phenyl)-N⁴,5-bis[2- (methyloxy)phenyl]-2,4- pyrimidinediamine | MS (ES+) m/e 514 [M + H]⁺ |
| 17 | 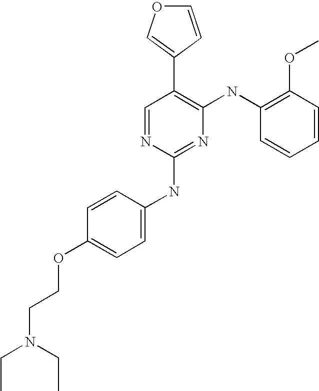 | N²-(4-{[2- (diethylamino)ethyl]oxy} phenyl)-5-(3-furanyl)-N⁴-[2- (methyloxy)phenyl]-2,4- pyrimidinediamine | MS (ES+) m/e 474 [M + H]⁺ |
| 18 | 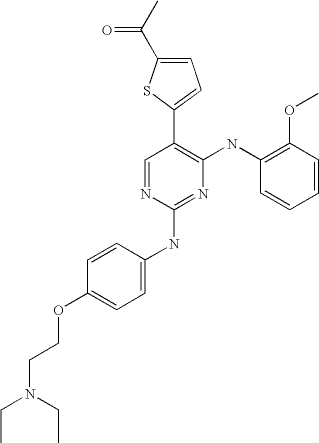 | 1-[5-(2-[(4-{[2- (diethylamino)ethyl]oxy} phenyl)amino]-4-{[2- (methyloxy)phenyl]amino}- 5-pyrimidinyl)-2- thienyl]ethanone | MS (ES+) m/e 532 [M + H]⁺ |
| 19 | 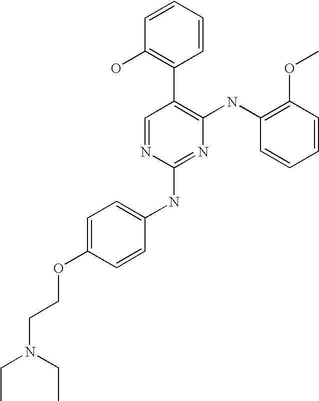 | 2-(2-[(4-{[2- (diethylamino)ethyl]oxy} phenyl)amino]-4-{[2- (methyloxy)phenyl]amino} 5-pyrimidinyl)phenol | MS (ES+) m/e 500 [M + H]⁺ |
| 20 | 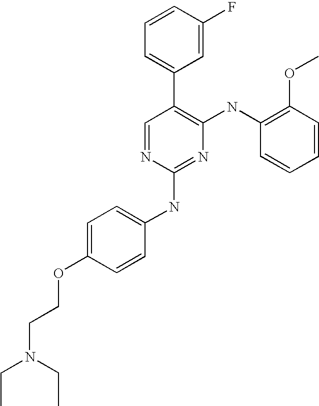 | N²-(4-{[2- (diethylamino)ethyl]oxy} phenyl)-5-(3-fluorophenyl)- N⁴-[2-(methyloxy)phenyl]- 2,4-pyrimidinediamine | MS (ES+) m/e 502 [M + H]⁺ |
| 21 | 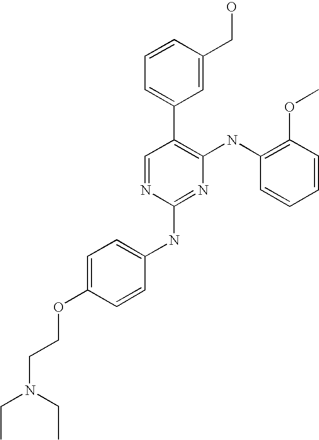 | [3-(2-[(4-{[2- (diethylamino)ethyl]oxy} phenyl)amino]-4-{[2- (methyloxy)phenyl]amino}- 5- pyrimidinyl)phenyl]methanol | MS (ES+) m/e 514 [M + H]⁺ |
| 22 | 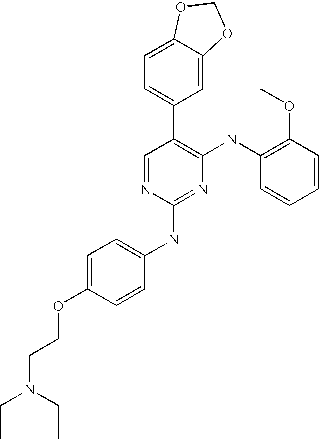 | 5-(1,3-benzodioxol-5-yl)- N²-(4-{[2- (diethylamino)ethyl]oxy} phenyl)-N⁴-[2- (methyloxy)phenyl]-2,4- pyrimidinediamine | MS (ES+) m/e 528 [M + H]⁺ |
| 23 | 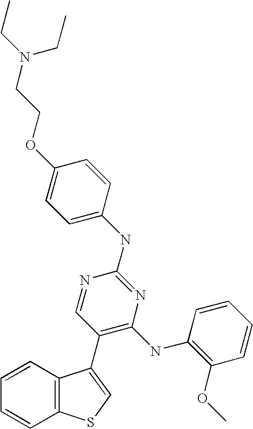 | 5-(1-benzothien-3-yl)-N²- (4-{[2- (diethylamino)ethyl]oxy} phenyl)-N⁴-[2- (methyloxy)phenyl]-2,4- pyrimidinediamine | MS (ES+) m/e 540 [M + H]⁺ |
| 24 | 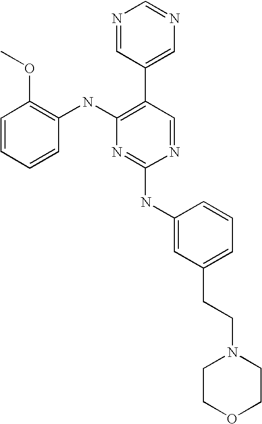 | N⁴-[2-(methyloxy)phenyl]- N²-{3-[2-(4- morpholinyl)ethyl]phenyl}- 5,5′-bipyrimidine-2,4- diamine | MS (ES+) m/e 484 [M + H]⁺ |
| 25 | 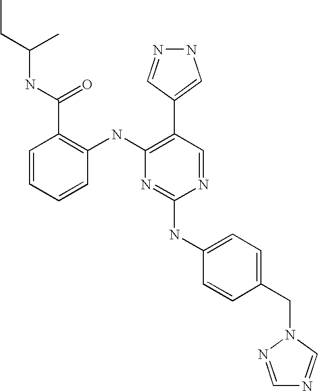 | N-(1-methylpropyl)-2-[(5- (1H-pyrazol-4-yl)-2-{[4- (1H-1,2,4-triazol-1- ylmethyl)phenyl]amino}-4- pyrimidinyl)amino]benzamide | MS (ES+) m/e 509 [M + H]⁺ |
| 26 | 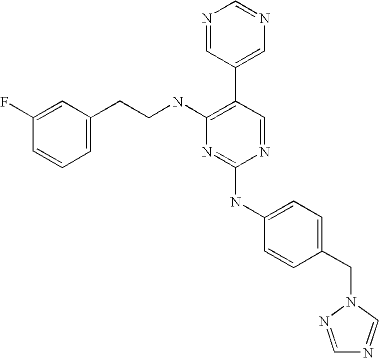 | N⁴-[2-(3- fluorophenyl)ethyl]-N²-[4- (1H-1,2,4-triazol-1- ylmethyl)phenyl]-5,5′- bipyrimidine-2,4-diamine | MS (ES+) m/e 468 [M + H]⁺ |
| 27 | 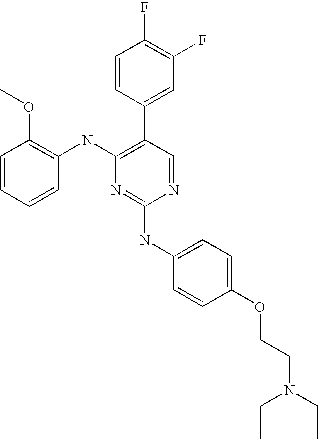 | N²-(4-{[2- (diethylamino)ethyl]oxy} phenyl)-5-(3,4- difluorophenyl)-N⁴-[2- (methyloxy)phenyl]-2,4- pyrimidinediamine | MS (ES+) m/e 520 [M + H]⁺ |
| 28 | 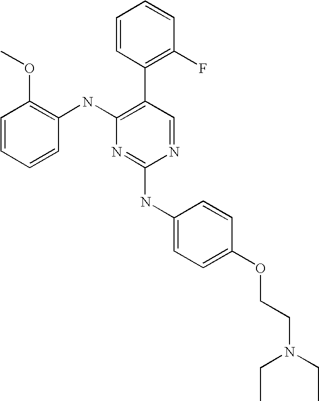 | N²-(4-{[2- (diethylamino)ethyl]oxy} phenyl)-5-(2-fluorophenyl)- N⁴-[2-(methyloxy)phenyl]- 2,4-pyrimidinediamine | MS (ES+) m/e 502 [M + H]⁺ |
| 29 | 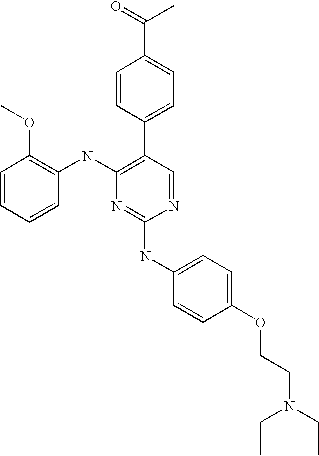 | 1-[4-(2-[(4-{[2- (diethylamino)ethyl]oxy} phenyl)amino]-4-{[2- (methyloxy)phenyl]amino}- 5- pyrimidinyl)phenyl]ethanone | MS (ES+) m/e 526 [M + H]⁺ |
| 30 | 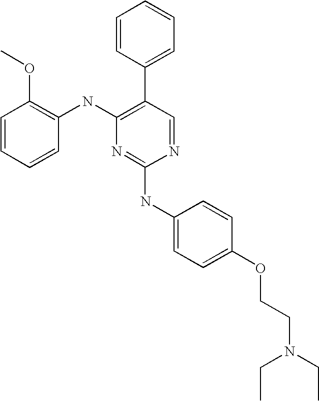 | N²-(4-{[2- (diethylamino)ethyl]oxy} phenyl)-N⁴-[2- (methyloxy)phenyl]-5- phenyl-2,4- pyrimidinediamine | MS (ES+) m/e 484 [M + H]⁺ |
| 31 | 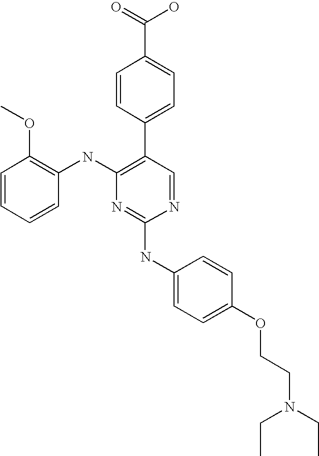 | 4-(2-[(4-{[2- (diethylamino)ethyl]oxy} phenyl)amino]-4-{[2- (methyloxy)phenyl]amino}- 5-pyrimidinyl)benzoic acid | MS (ES+) m/e 528 [M + H]⁺ |
| 32 | 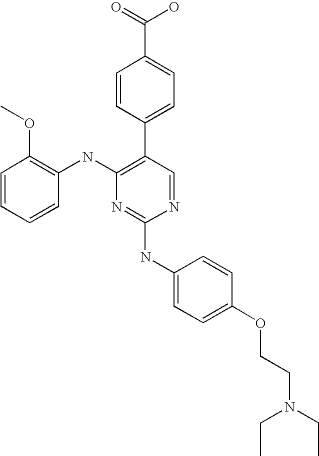 | 4-(2-[(4-{[2- (diethylamino)ethyl]oxy} phenyl)amino]-4-{[2- (methyloxy)phenyl]amino}- 5-pyrimidinyl)benzoic acid | MS (ES+) m/e 468 [M + H]⁺ |
| 33 | 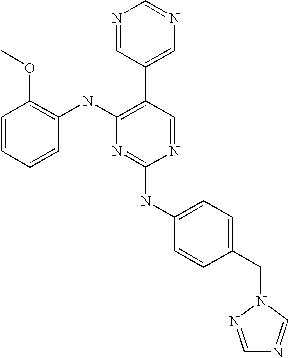 | N⁴-[2-(methyloxy)phenyl]- N²-[4-(1H-1,2,4-triazol-1- ylmethyl)phenyl]-5,5′- bipyrimidine-2,4-diamine | MS (ES+) m/e 452 [M + H]⁺ |
| 34 | 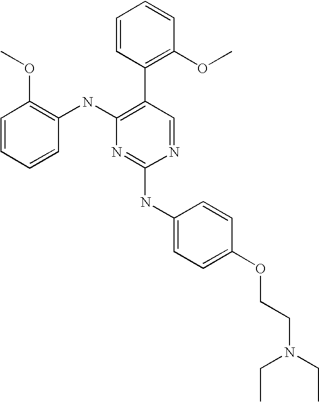 | N²-(4-{[2- (diethylamino)ethyl]oxy} phenyl)-N⁴,5-bis[2- (methyloxy)phenyl]-2,4- pyrimidinediamine | MS (ES+) m/e 514 [M + H]⁺ |
| 35 | 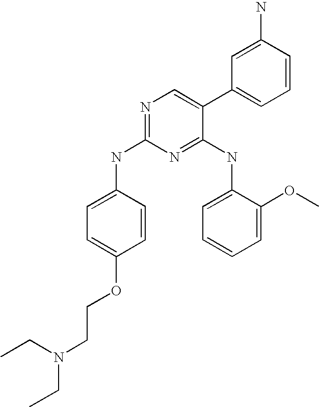 | 5-(3-aminophenyl)-N²-(4- {[2- (diethylamino)ethyl]oxy} phenyl)-N⁴-[2- (methyloxy)phenyl]-2,4- pyrimidinediamine | MS (ES+) m/e 499 [M + H]⁺ |
| 36 | 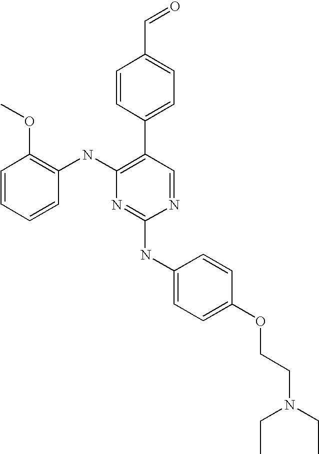 | 4-(2-[(4-{[2- (diethylamino)ethyl]oxy} phenyl)amino]-4-{[2- (methyloxy)phenyl]amino}- 5- pyrimidinyl)benzaldehyde | MS (ES+) m/e 512 [M + H]⁺ |
| 37 | 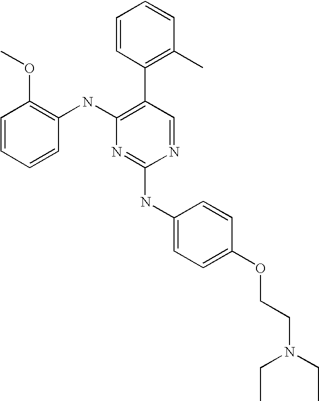 | N²-(4-{[2- (diethylamino)ethyl]oxy} phenyl)-N⁴-[2- (methyloxy)phenyl]-5-(2- methylphenyl)-2,4- pyrimidinediamine | MS (ES+) m/e 498 [M + H]⁺ |
| 38 | 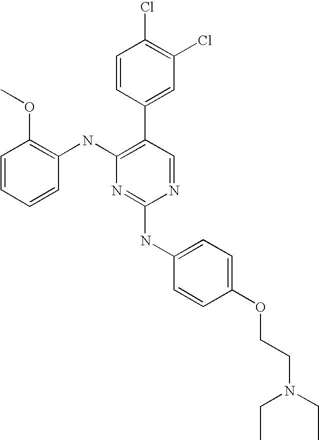 | 5-(3,4-dichlorophenyl)-N²- (4-{[2- (diethylamino)ethyl]oxy} phenyl)-N⁴-[2- (methyloxy)phenyl]-2,4- pyrimidinediamine | MS (ES+) m/e 552 [M + H]⁺ |
| 39 | 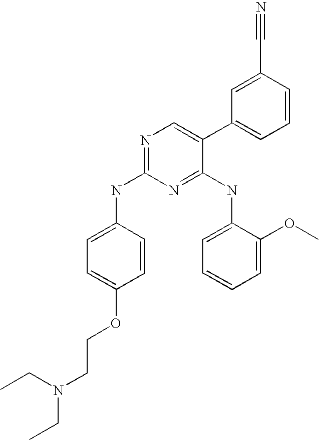 | 3-(2-[(4-{[2- (diethylamino)ethyl]oxy} phenyl)amino]-4-{[2- (methyloxy)phenyl]amino}- 5-pyrimidinyl)benzonitrile | MS (ES+) m/e 509 [M + H]⁺ |
| 40 | 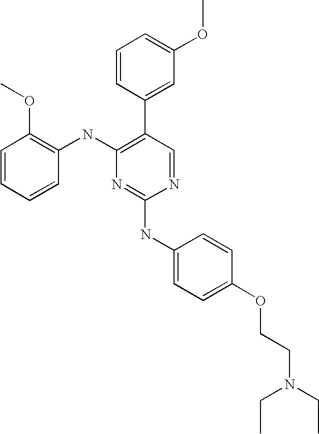 | N²-(4-{[2- (diethylamino)ethyl]oxy} phenyl)-N⁴-[2- (methyloxy)phenyl]-5-[3- (methyloxy)phenyl]-2,4- pyrimidinediamine | MS (ES+) m/e 514 [M + H]⁺ |
| 41 | 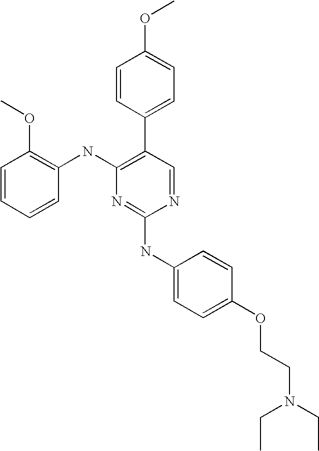 | N²-(4-{[2- (diethylamino)ethyl]oxy} phenyl)-N⁴-[2- (methyloxy)phenyl]-5-[4- (methyloxy)phenyl]-2,4- pyrimidinediamine | MS (ES+) m/e 514 [M + H]⁺ |
| 42 | 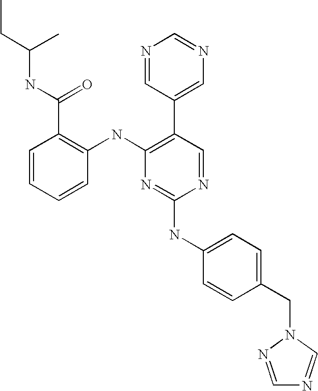 | N-(1-methylpropyl)-2-[(2- {[4-(1H-1,2,4-triazol-1- ylmethyl)phenyl]amino}- 5,5′-bipyrimidin-4- yl)amino]benzamide | MS (ES+) m/e 521 [M + H]⁺ |
| 43 | 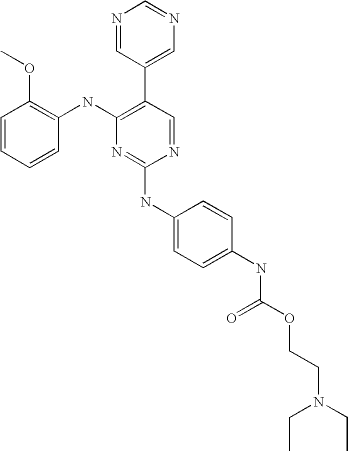 | 2-(diethylamino)ethyl {4- [(4-{[2- (methyloxy)phenyl]amino}- 5,5′-bipyrimidin-2- yl)amino]phenyl}carbamate | MS (ES+) m/e 529 [M + H]⁺ |
| 44 | 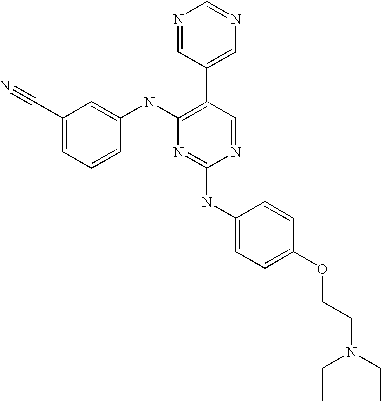 | 3-({2-[(4-{[2- (diethylamino)ethyl]oxy} phenyl)amino]-5,5′- bipyrimidin-4- yl}amino)benzonitrile | MS (ES+) m/e 481 [M + H]⁺ |
| 45 | 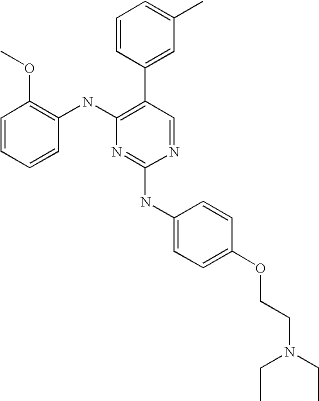 | N²-(4-{[2- (diethylamino)ethyl]oxy} phenyl)-N⁴-[2- (methyloxy)phenyl]-5-(3- methylphenyl)-2,4- pyrimidinediamine | MS (ES+) m/e 498 [M + H]⁺ |
| 46 | 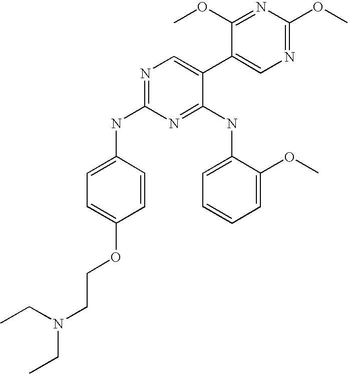 | N²-(4-{[2- (diethylamino)ethyl]oxy} phenyl)-2′,4′-bis(methyloxy)- N⁴-[2-(methyloxy)phenyl]- 5,5′-bipyrimidine-2,4- diamine | MS (ES+) m/e 545 [M + H]⁺ |
| 47 | 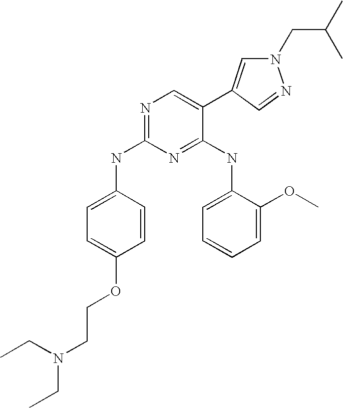 | N²-(4-{[2- (diethylamino)ethyl]oxy} phenyl)-N⁴-[2- (methyloxy)phenyl]-5-[1-(2- methylpropyl)-1H-pyrazol- 4-yl]-2,4-pyrimidinediamine | MS (ES+) m/e 530 [M + H]⁺ |
| 48 | 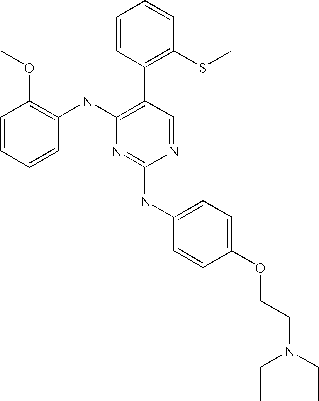 | N²-(4-{[2- (diethylamino)ethyl]oxy} phenyl)-N⁴-[2- (methyloxy)phenyl]-5-[2- (methylthio)phenyl]-2,4- pyrimidinediamine | MS (ES+) m/e 530 [M + H]⁺ |
| 49 | 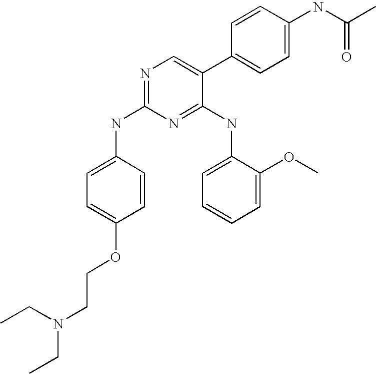 | N-[4-(2-[(4-{[2- (diethylamino)ethyl]oxy} phenyl)amino]-4-{[2- (methyloxy)phenyl]amino}- 5- pyrimidinyl)phenyl]acetamide | MS (ES+) m/e 541 [M + H]⁺ |
| 50 | 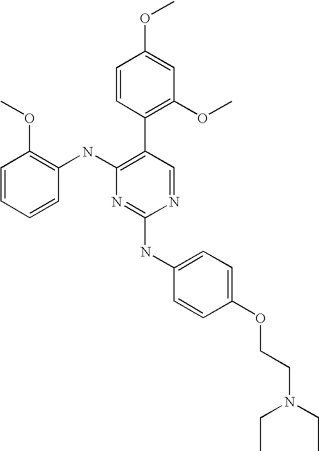 | 5-[2,4- bis(methyloxy)phenyl]-N²- (4-{[2- (diethylamino)ethyl]oxy} phenyl)-N⁴-[2- (methyloxy)phenyl]-2,4- pyrimidinediamine | MS (ES+) m/e 544 [M + H]⁺ |
| 51 | 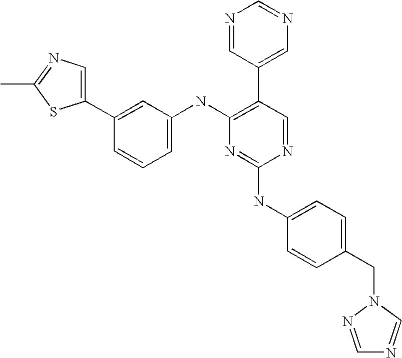 | N⁴-[3-(2-methyl-1,3-thiazol- 5-yl)phenyl]-N²-[4-(1H- 1,2,4-triazol-1- ylmethyl)phenyl]-5,5′- bipyrimidine-2,4-diamine | MS (ES+) m/e 519 [M + H]⁺ |
| 52 | 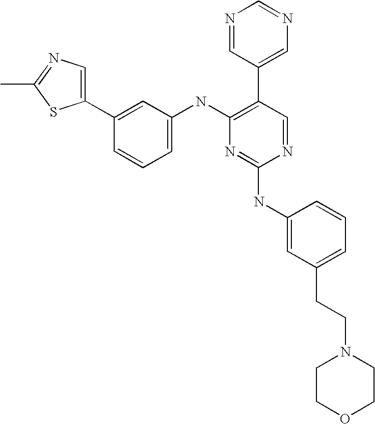 | N⁴-[3-(2-methyl-1,3-thiazol- 5-yl)phenyl]-N²-{3-[2-(4- morpholinyl)ethyl]phenyl}- 5,5′-bipyrimidine-2,4- diamine | MS (ES+) m/e 551 [M + H]⁺ |
| 53 | 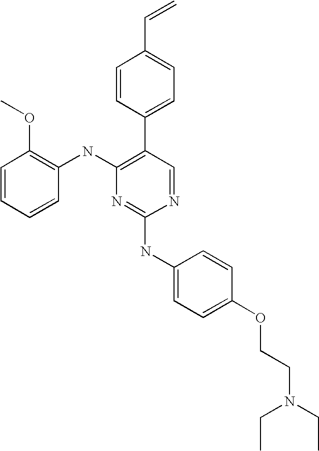 | N²-(4-{[2- (diethylamino)ethyl]oxy} phenyl)-5-(4-ethenylphenyl)- N⁴-[2-(methyloxy)phenyl]- 2,4-pyrimidinediamine | MS (ES+) m/e 510 [M + H]⁺ |
| 54 | 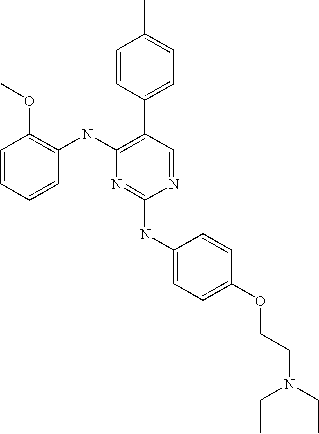 | N²-(4-{[2- (diethylamino)ethyl]oxy} phenyl)-N⁴-[2- (methyloxy)phenyl]-5-(4- methylphenyl)-2,4- pyrimidinediamine | MS (ES+) m/e 498 [M + H]⁺ |
| 55 | 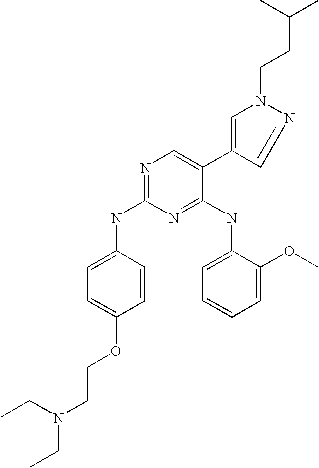 | N²-(4-{[2- (diethylamino)ethyl]oxy} phenyl)-5-[1-(3-methylbutyl)- 1H-pyrazol-4-yl]-N⁴-[2- (methyloxy)phenyl]-2,4- pyrimidinediamine | MS (ES+) m/e 544 [M + H]⁺ |
| 56 | 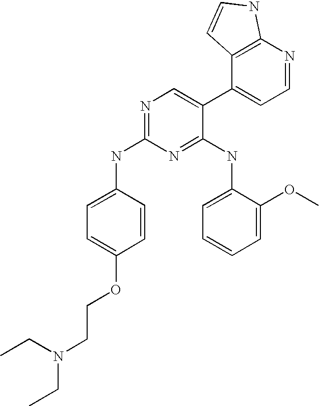 | N²-(4-{[2- (diethylamino)ethyl]oxy} phenyl)-N⁴-[2- (methyloxy)phenyl]-5-(1H- pyrrolo[2,3-b]pyridin-4-yl)- 2,4-pyrimidinediamine | MS (ES+) m/e 524 [M + H]⁺ |
| 57 | 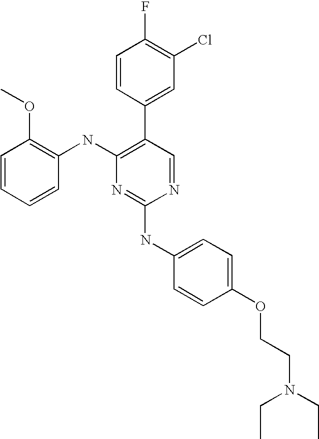 | 5-(3-chloro-4- fluorophenyl)-N²-(4-{[2- (diethylamino)ethyl]oxy} phenyl)-N⁴-[2- (methyloxy)phenyl]-2,4- pyrimidinediamine | MS (ES+) m/e 536 [M + H]⁺ |
| 58 | 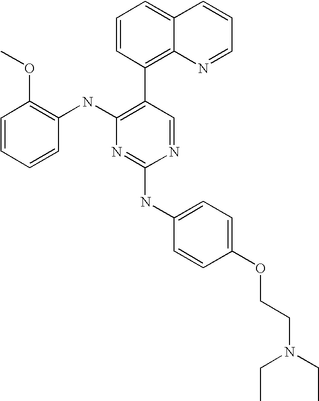 | N²-(4-{[2- (diethylamino)ethyl]oxy} phenyl)-N⁴-[2- (methyloxy)phenyl]-5-(8- quinolinyl)-2,4- pyrimidinediamine | MS (ES+) m/e 535 [M + H]⁺ |
| 59 | 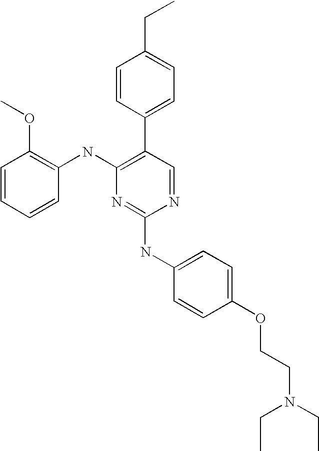 | N²-(4-{[2- (diethylamino)ethyl]oxy} phenyl)-5-(4-ethylphenyl)-N⁴- [2-(methyloxy)phenyl]-2,4- pyrimidinediamine | MS (ES+) m/e 512 [M + H]⁺ |
| 60 | 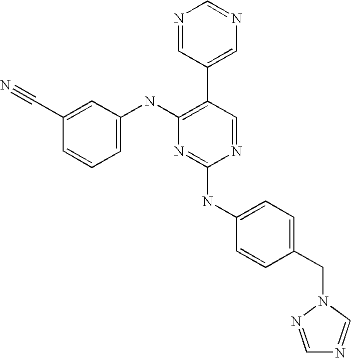 | 3-[(2-{[4-(1H-1,2,4-triazol- 1-ylmethyl)phenyl]amino}- 5,5′-bipyrimidin-4- yl)amino]benzonitrile | MS (ES+) m/e 447 [M + H]⁺ |
| 61 | 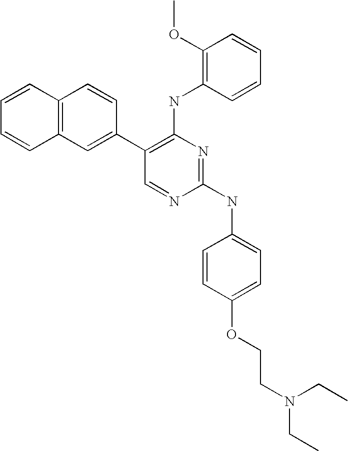 | N²-(4-{[2- (diethylamino)ethyl]oxy} phenyl)-N⁴-[2- (methyloxy)phenyl]-5-(2- naphthalenyl)-2,4- pyrimidinediamine | MS (ES+) m/e 534 [M + H]⁺ |
| 62 | 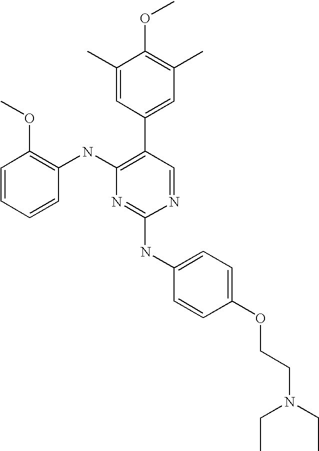 | N²-(4-{[2- (diethylamino)ethyl]oxy} phenyl)-5-[3,5-dimethyl-4- (methyloxy)phenyl]-N⁴-[2- (methyloxy)phenyl]-2,4- pyrimidinediamine | MS (ES+) m/e 542 [M + H]⁺ |
| 63 | 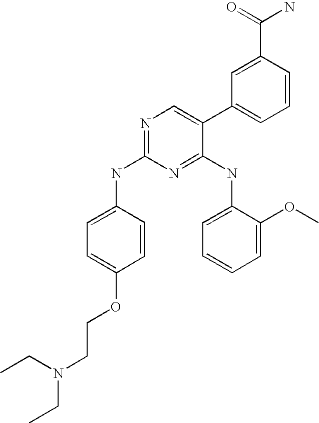 | 3-(2-[(4-{[2- (diethylamino)ethyl]oxy} phenyl)amino]-4-{[2- (methyloxy)phenyl]amino}- 5-pyrimidinyl)benzamide | MS (ES+) m/e 527 [M + H]⁺ |
| 64 | 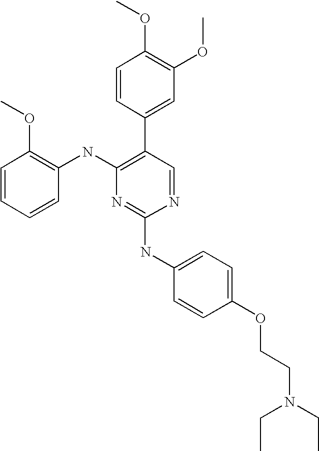 | 5-[3,4- bis(methyloxy)phenyl]-N²- (4-{[2- (diethylamino)ethyl]oxy} phenyl)-N⁴-[2- (methyloxy)phenyl]-2,4- pyrimidinediamine | MS (ES+) m/e 544 [M + H]⁺ |
| 65 | 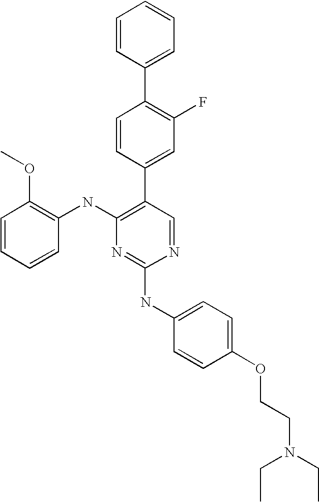 | N²-(4-{[2- (diethylamino)ethyl]oxy} phenyl)-5-(2-fluoro-4- biphenylyl)-N⁴-[2- (methyloxy)phenyl]-2,4- pyrimidinediamine | MS (ES+) m/e 578 [M + H]⁺ |
| 66 | 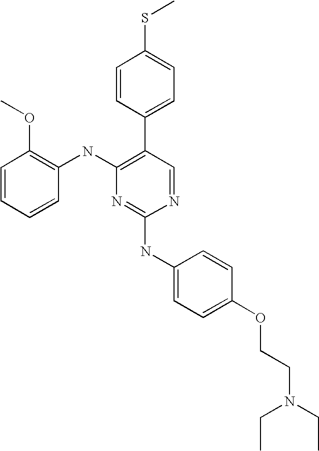 | N²-(4-{[2- (diethylamino)ethyl]oxy} phenyl)-N⁴-[2- (methyloxy)phenyl]-5-[4- (methylthio)phenyl]-2,4- pyrimidinediamine | MS (ES+) m/e 530 [M + H]⁺ |
| 67 | 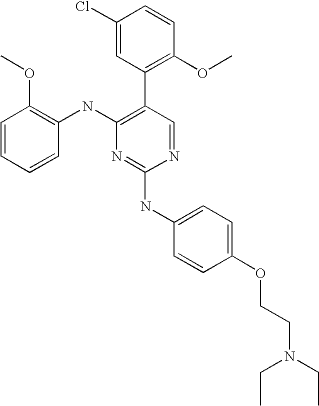 | 5-[5-chloro-2- (methyloxy)phenyl]-N²-(4- {[2- (diethylamino)ethyl]oxy} phenyl)-N⁴-[2- (methyloxy)phenyl]-2,4- pyrimidinediamine | MS (ES+) m/e 548 [M + H]⁺ |
| 68 | 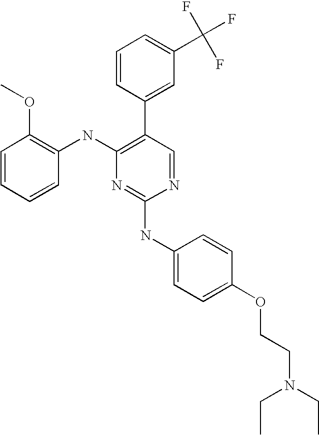 | N²-(4-{[2- (diethylamino)ethyl]oxy} phenyl)-N⁴-[2- (methyloxy)phenyl]-5-[3- (trifluoromethyl)phenyl]- 2,4-pyrimidinediamine | MS (ES+) m/e 551 [M + H]⁺ |
| 69 | 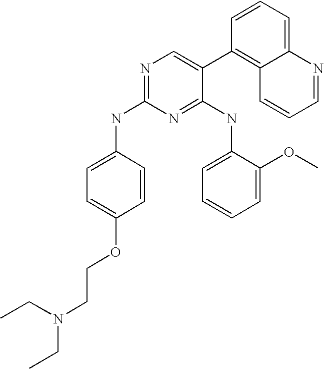 | N²-(4-{[2- (diethylamino)ethyl]oxy} phenyl)-N⁴-[2- (methyloxy)phenyl]-5-(5- quinolinyl)-2,4- pyrimidinediamine | MS (ES+) m/e 535 [M + H]⁺ |
| 70 | 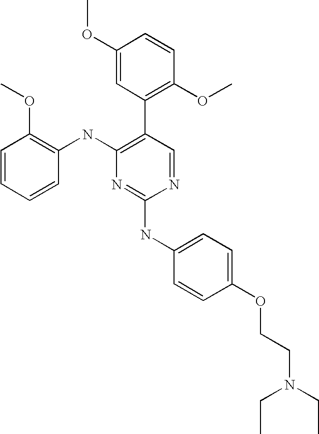 | 5-[2,5- bis(methyloxy)phenyl]-N²- (4-{[2- (diethylamino)ethyl]oxy} phenyl)-N⁴-[2- (methyloxy)phenyl]-2,4- pyrimidinediamine | MS (ES+) m/e 544 [M + H]⁺ |
| 71 | 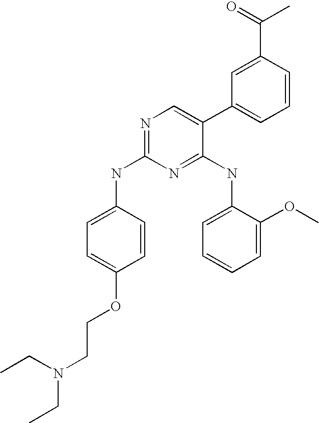 | 1-[3-(2-[(4-{[2- (diethylamino)ethyl]oxy} phenyl)amino]-4-{[2- (methyloxy)phenyl]amino}- 5- pyrimidinyl)phenyl]ethanone | MS (ES+) m/e 526 [M + H]⁺ |
| 72 | 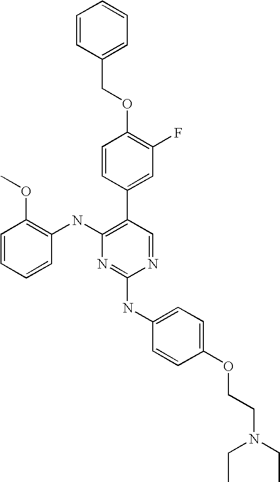 | N²-(4-{[2- (diethylamino)ethyl]oxy} phenyl)-5-{3-fluoro-4- [(phenylmethyl)oxy]phenyl}- N⁴-[2-(methyloxy)phenyl]- 2,4-pyrimidinediamine | MS (ES+) m/e 608 [M + H]⁺ |
| 73 | 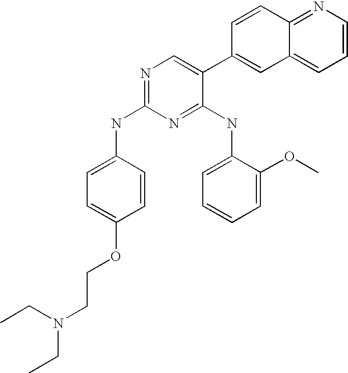 | N²-(4-{[2- (diethylamino)ethyl]oxy} phenyl)-N⁴-[2- (methyloxy)phenyl]-5-(6- quinolinyl)-2,4- pyrimidinediamine | MS (ES+) m/e 535 [M + H]⁺ |
| 74 | 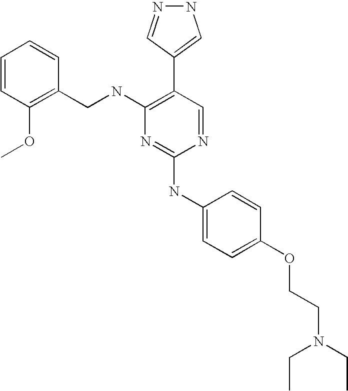 | N²-(4-{[2- (diethylamino)ethyl]oxy} phenyl)-N⁴-{[2- (methyloxy)phenyl]methyl}- 5-(1H-pyrazol-4-yl)-2,4- pyrimidinediamine | MS (ES+) m/e 488 [M + H]⁺ |
| 75 | 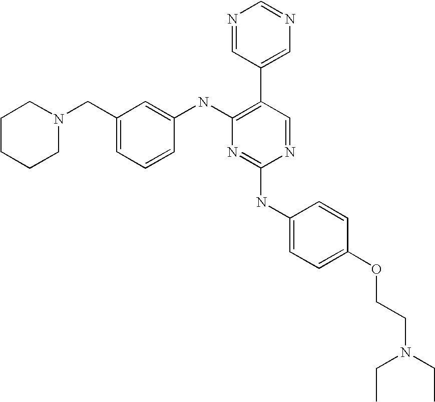 | N²-(4-{[2- (diethylamino)ethyl]oxy} phenyl)-N⁴-[3-(1- piperidinylmethyl)phenyl]- 5,5′-bipyrimidine-2,4- diamine | MS (ES+) m/e 553 [M + H]⁺ |
| 76 | 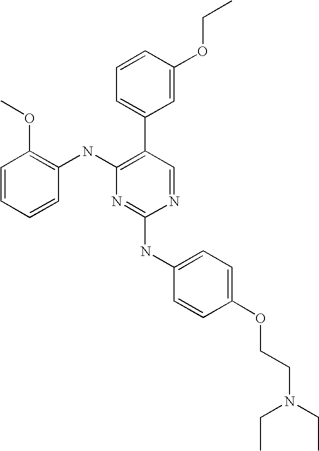 | N²-(4-{[2- (diethylamino)ethyl]oxy} phenyl)-5-[3- (ethyloxy)phenyl]-N⁴-[2- (methyloxy)phenyl]-2,4- pyrimidinediamine | MS (ES+) m/e 528 [M + H]⁺ |
| 77 | 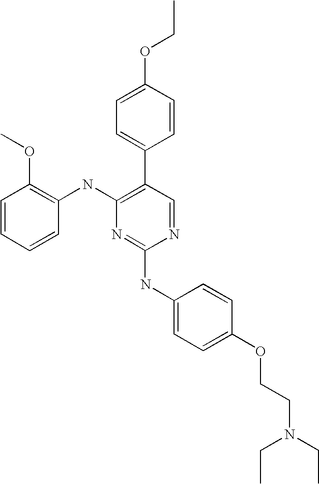 | N²-(4-{[2- (diethylamino)ethyl]oxy} phenyl)-5-[4- (ethyloxy)phenyl]-N⁴-[2- (methyloxy)phenyl]-2,4- pyrimidinediamine | MS (ES+) m/e 528 [M + H]⁺ |
| 78 | 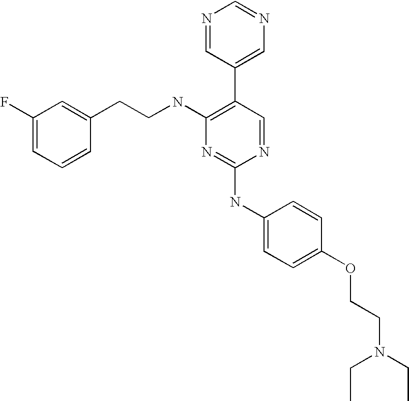 | N²-(4-{[2- (diethylamino)ethyl]oxy} phenyl)-N⁴-[2-(3- fluorophenyl)ethyl]-5,5′- bipyrimidine-2,4-diamine | MS (ES+) m/e 502 [M + H]⁺ |
| 79 | 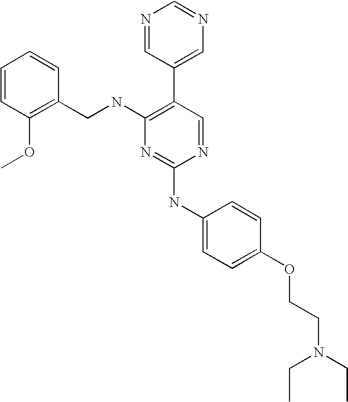 | N²-(4-{[2- (diethylamino)ethyl]oxy} phenyl)-N⁴-{[2- (methyloxy)phenyl]methyl}- 5,5′-bipyrimidine-2,4- diamine | MS (ES+) m/e 500 [M + H]⁺ |
| 80 | 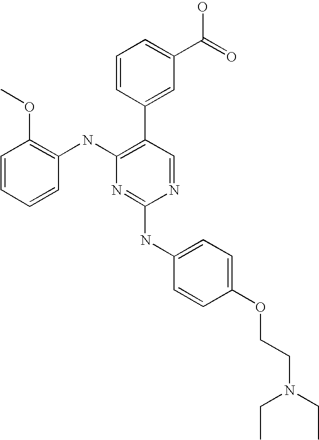 | 3-(2-[(4-{[2- (diethylamino)ethyl]oxy} phenyl)amino]-4-{[2- (methyloxy)phenyl]amino}- 5-pyrimidinyl)benzoic acid | MS (ES+) m/e 528 [M + H]⁺ |
| 81 | 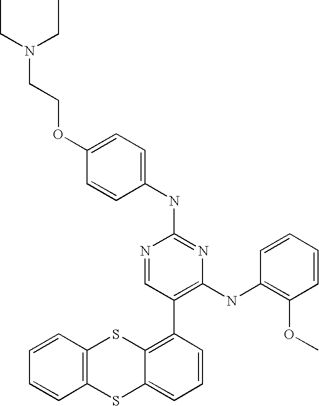 | N²-(4-{[2- (diethylamino)ethyl]oxy} phenyl)-N⁴-[2- (methyloxy)phenyl]-5-(1- thianthrenyl)-2,4- pyrimidinediamine | MS (ES+) m/e 622 [M + H]⁺ |
| 82 | 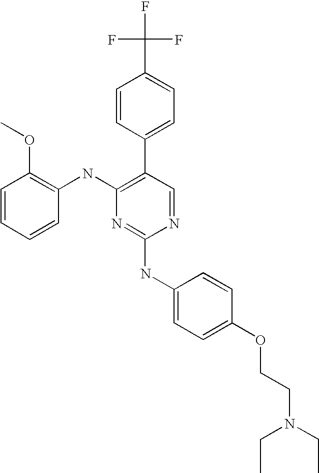 | N²-(4-{[2- (diethylamino)ethyl]oxy} phenyl)-N⁴-[2- (methyloxy)phenyl]-5-[4- (trifluoromethyl)phenyl]- 2,4-pyrimidinediamine | MS (ES+) m/e 552 [M + H]⁺ |
| 83 | 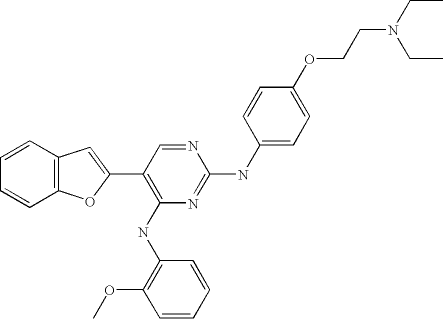 | 5-(1-benzofuran-2-yl)-N²- (4-{[2- (diethylamino)ethyl]oxy} phenyl)-N⁴-[2- (methyloxy)phenyl]-2,4- pyrimidinediamine | MS (ES+) m/e 524 [M + H]⁺ |
| 84 | 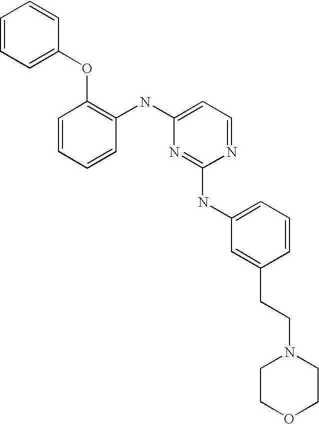 | N²-{3-[2-(4- morpholinyl)ethyl]phenyl}- N⁴-[2-(phenyloxy)phenyl]- 2,4-pyrimidinediamine | MS (ES+) m/e 468 [M + H]⁺ |
| 85 | 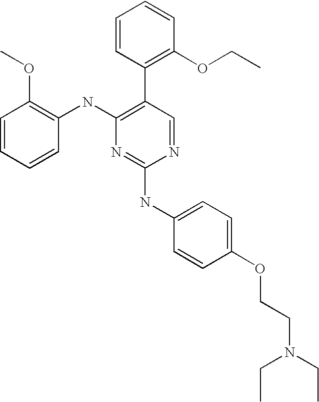 | N²-(4-{[2- (diethylamino)ethyl]oxy} phenyl)-5-[2- (ethyloxy)phenyl]-N⁴-[2- (methyloxy)phenyl]-2,4- pyrimidinediamine | MS (ES+) m/e 528 [M + H]⁺ |
| 86 | 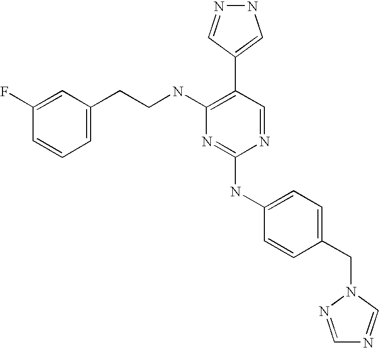 | N⁴-[2-(3- fluorophenyl)ethyl]-5-(1H- pyrazol-4-yl)-N²-[4-(1H- 1,2,4-triazol-1- ylmethyl)phenyl]-2,4- pyrimidinediamine | MS (ES+) m/e 456 [M + H]⁺ |
| 87 | 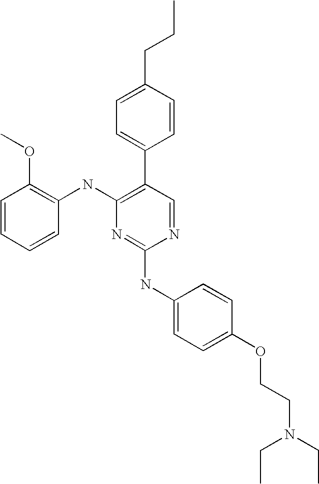 | N²-(4-{[2- (diethylamino)ethyl]oxy} phenyl)-N⁴-[2- (methyloxy)phenyl]-5-(4- propylphenyl)-2,4- pyrimidinediamine | MS (ES+) m/e 525 [M + H]⁺ |
| 88 | 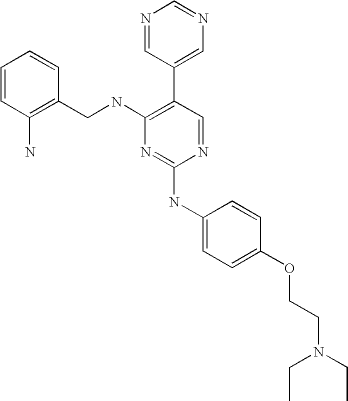 | N⁴-[(2- aminophenyl)methyl]-N²- (4-{[2- (diethylamino)ethyl]oxy} phenyl)-5,5′-bipyrimidine-2,4- diamine | MS (ES+) m/e 485 [M + H]⁺ |
| 89 | 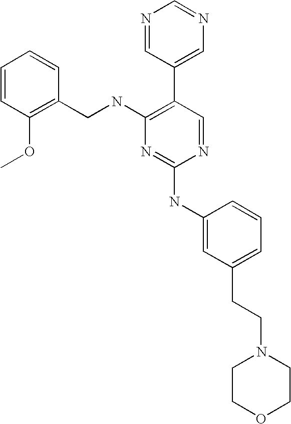 | N⁴-{[2- (methyloxy)phenyl]methyl}- N²-{3-[2-(4- morpholinyl)ethyl]phenyl}- 5,5′-bipyrimidine-2,4- diamine | MS (ES+) m/e 498 [M + H]⁺ |
| 90 | 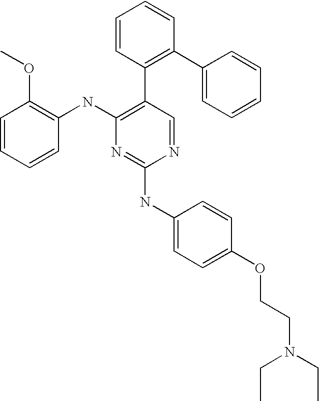 | 5-(2-biphenylyl)-N²-(4-{[2- (diethylamino)ethyl]oxy} phenyl)-N⁴-[2- (methyloxy)phenyl]-2,4- pyrimidinediamine | MS (ES+) m/e 560 [M + H]⁺ |
| 91 | 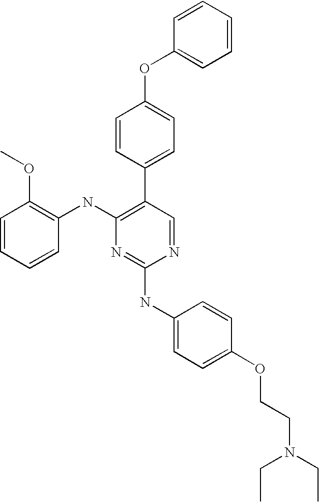 | 5-(2-biphenylyl)-N²-(4-{[2- (diethylamino)ethyl]oxy} phenyl)-N⁴-[2- (methyloxy)phenyl]-2,4- pyrimidinediamine | MS (ES+) m/e 576 [M + H]⁺ |
| 92 | 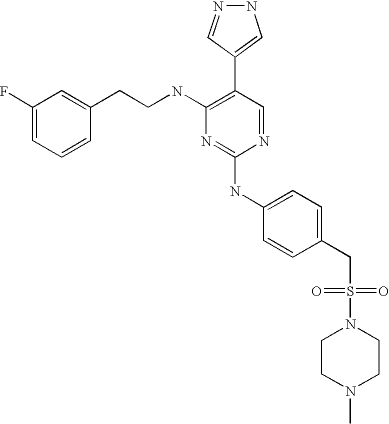 | N⁴-[2-(3- fluorophenyl)ethyl]-N²-(4- {[(4-methyl-1- piperazinyl)sulfonyl]methyl} phenyl)-5-(1H-pyrazol-4- yl)-2,4-pyrimidinediamine | MS (ES+) m/e 551 [M + H]⁺ |
| 93 | 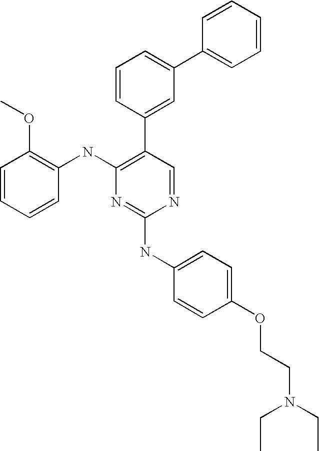 | 5-(3-biphenylyl)-N²-(4-{[2- (diethylamino)ethyl]oxy} phenyl)-N⁴-[2- (methyloxy)phenyl]-2,4- pyrimidinediamine | MS (ES+) m/e 560 [M + H]⁺ |
| 94 | 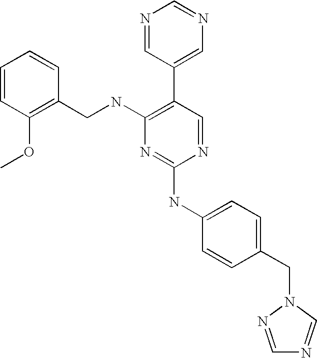 | N⁴-{[2- (methyloxy)phenyl]methyl}- N²-[4-(1H-1,2,4-triazol-1- ylmethyl)phenyl]-5,5′- bipyrimidine-2,4-diamine | MS (ES+) m/e 466 [M + H]⁺ |
| 95 | 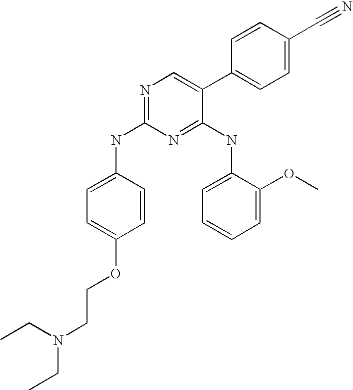 | 4-(2-[(4-{[2- (diethylamino)ethyl]oxy} phenyl)amino]-4-{[2- (methyloxy)phenyl]amino}- 5-pyrimidinyl)benzonitrile | MS (ES+) m/e 509 [M + H]⁺ |
| 96 | 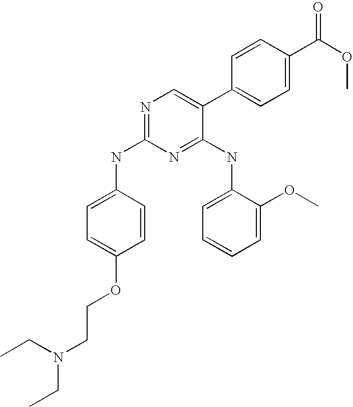 | methyl 4-(2-[(4-{[2- (diethylamino)ethyl]oxy} phenyl)amino]-4-{[2- (methyloxy)phenyl]amino}- 5-pyrimidinyl)benzoate | MS (ES+) m/e 542 [M + H]⁺ |
| 97 | 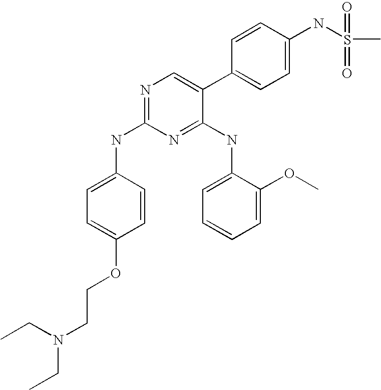 | methyl 4-(2-[(4-{[2- (diethylamino)ethyl]oxy} phenyl)amino]-4-{[2- (methyloxy)phenyl]amino}- 5-pyrimidinyl)benzoate | MS (ES+) m/e 577 [M + H]⁺ |
| 98 | 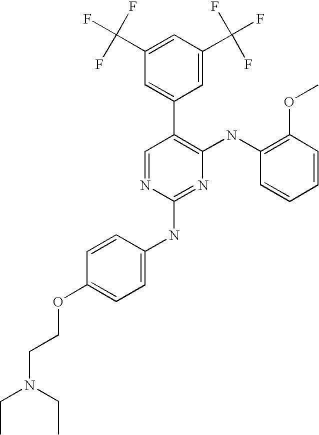 | 5-[3,5- bis(trifluoromethyl)phenyl]- N²-(4-{[2- (diethylamino)ethyl]oxy} phenyl)-N⁴-[2- (methyloxy)phenyl]-2,4- pyrimidinediamine | MS (ES+) m/e 620 [M + H]⁺ |
| 99 | 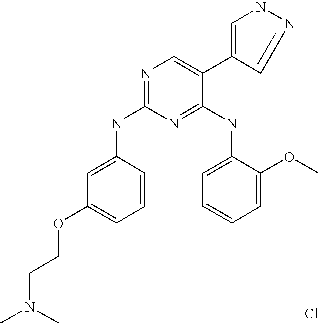 | N²-(3-{[2- (dimethylamino)ethyl]oxy}phenyl)- N⁴-[2-(methyloxy)phenyl]-5-(1H- pyrazol-4-yl)-2,4- pyrimidinediamine hydrochloride | MS (ES+) m/e 446.0 [M + H]+. |

### In Vitro Assay for Wee1 Inhibitory Activity

Inhibition of Wee1 kinase activity was determined using recombinantly-expressed human Wee1 kinase with amino acids 1-13 deleted. The substrate for the assay was a chemically biotinylated recombinantly-expressed CDK1 (cdc2/cyclinB) for which the coding sequence had been modified to eliminate kinase activity (K33R). The kinase activity of Wee1 was quantified by time-resolved fluorescence resonance energy transfer technology using an europium-labeled anti-phosphotyrosine antibody and strepavidin-labeled allophycocyanin. The test compounds were typically assayed over an eleven point dilution range with a concentration in the assay of 10 uM to 0.2 nM, in 3-fold dilutions. This assay was used to calculate a pIC50 for all of the compounds described in Examples 3-99. All of the tested compounds had a pIC50≧5.0.

### Cell Assay for Wee1 Inhibitory Activity

Wee1 inhibitory activity can be measured using a cell-based ELISA assay.

Hela cells are synchronized using aphidicolin, which blocks the entry of cells into S-phase. Cells in G2-M transition phase are then obtained by releasing the cells from aphidicolin treatment for approximately 7-9 hrs. The phosphorylation level of the Wee1 target cdc2 may then be measured by sandwich ELISA using an anti-cdc2 antibody and an anti-phospho-cdc2(Tyr15) antibody. This cell assay was used to calculate a pIC50 for the compounds described in Examples 3, 4, 6-8, 10-12, 15, 17, 18, 21, 23, 25-27, 33, 35, 39, 43, 44, 51, 63, and 99. The compounds shown in examples 3, 4, 6-8, 10-12, 21, 25, 26, 33, 35, 43, 44, 51, 63, and 99 had a pIC50≧5.0 in this assay.

Those of skill in the art will recognize that activities for enzyme activity such as the in vitro HTRF assay and the cell assay described above are subject to variability. Accordingly, it is to be understood that the values for the pIC50s recited above are exemplary only.

What is claimed is:

1. A compound of Formula (I): 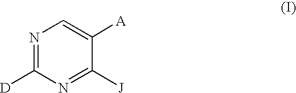
or a salt thereof, wherein:
A is selected from —H, aryl optionally substituted with at least one R group, and heteroaryl optionally substituted with at least one R^{a} group;
Each R is independently selected from the group consisting of halo, —OH, —NH₂, —CN, C₁-C₃ alkoxy, aryloxy, aralkoxy, —CHO, —C(O)R″, —C(O)OR″, —C(O)OH, —C(O)H, —C(O)NR′R″, —NO₂, —N(H)C(O)R″, —N(H)S(O)₂R″, C₁-C₃ alkyl, C₁-C₃ hydroxyalkyl, C₁-C₃ haloalkyl, C₂-C₄ alkenyl, —(CH₂)ₒX, —SR″, and aryl;
o is 0 or 1;
Each R^{a} is independently selected from the group consisting of C₁-C₆ alkyl, C₁-C₃ alkoxy, C(O)R″, and aralkyl;
J is selected from 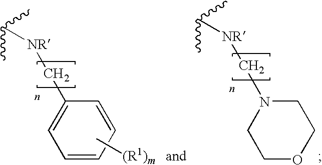
m is or 0 or 1;
n is 0, 1, or 2;
R¹ is halo, —CN, —NH₂, C₁-C₃ alkoxy, aryloxy, —C(O)N(H)R′, —C(O)OR″, heteroaryl optionally substituted with at least one C₁-C₃ alkyl, or (CH₂)_{q}X;
q is 0 or 1;
D is: 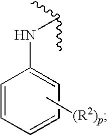
R² is selected from the group consisting of —O(CH₂)ₒNR′R″, —N(H)C(O)O(CH₂)ₒNR′R″, —(CH₂)ₒX, and —CH₂S(O)₂X;
p is 1;
o is 1 or 2;
R′ is —H or C₁-C₄ alkyl;
R″ is C₁-C₄ alkyl; and
X is heterocyclyl or heteroaryl.

2. A compound according to claim 1 wherein A is heteroaryl.

3. A compound according to claim 1 wherein J is: 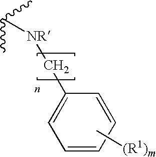
wherein:
m is or 0 or 1;
n is 0, 1, or 2
R¹ is selected from halo, —CN, —NH₂, C₁-C₃ alkoxy, aryloxy, —C(O)N(H)R′, —C(O)OR″, heteroaryl optionally substituted with at least one C₁-C₃ alkyl, and —(CH₂)_{q}X; and
R′ is —H, C₁-C₄ alkyl.

4. A compound according to claim 3 wherein m is 1, n is 0, R¹ is C₁-C₃ alkoxy, and R′ is —H.

5. A compound according to claim 1 wherein R² is —O(CH₂)ₒNR′R″, p is 1, o is 2, R′ is —H, and R″ is C₁-C₄ alkyl.

6. A compound as claimed in claim 1, wherein said compound is selected from the group consisting of:
N²-(4-{[2-(diethylamino)ethyl]oxy}phenyl)-N⁴-[2-(methyloxy)phenyl]-5-(1H-pyrazol-4-yl)-2,4-pyrimidinediamine;
N²-(4-{[2-(diethylamino)ethyl]oxy}phenyl)-5-(1H-indazol-5-yl)-N⁴-[2-(methyloxy)phenyl]-2,4-pyrimidinediamine;
[4-(2-[(4-{[2-(diethylamino)ethyl]oxy}phenyl)amino]-4-{[2-(methyloxy)phenyl]amino}-5-pyrimidinyl)phenyl]methanol;
N²-(4-{[2-(diethylamino)ethyl]oxy}phenyl)-N⁴-[2-(methyloxy)phenyl]-5-[5-(methyloxy)-3-pyridinyl]-2,4-pyrimidinediamine;
4-(2-[(4-{[2-(diethylamino)ethyl]oxy}phenyl)amino]-4-{[2-(methyloxy)phenyl]amino}-5-pyrimidinyl)phenol;
N²-(4-{[2-(diethylamino)ethyl]oxy}phenyl)-N⁴-[2-(methyloxy)phenyl]-5,5′-bipyrimidine-2,4-diamine;
N²-(4-{[2-(diethylamino)ethyl]oxy}phenyl)-N⁴-[2-(methyloxy)phenyl]-5-(3-pyridinyl)-2,4-pyrimidinediamine;
N²-(4-{[2-(diethylamino)ethyl]oxy}phenyl)-N⁴-[2-(methyloxy)phenyl]-5-(3-quinolinyl)-2,4-pyrimidinediamine;
5-(2-chlorophenyl)-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[1-(phenylmethyl)-1H-pyrazol-4-yl]-2,4-pyrimidinediamine;
N²-(4-{[2-(diethylamino)ethyl]oxy}phenyl)-N⁴-[2-(methyloxy)phenyl]-5-(3-quinolinyl)-2,4-pyrimidinediamine;
N²-(4-{[2-(diethylamino)ethyl]oxy}phenyl)-5-(3,5-dimethyl-1H-pyrazol-4-yl)-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)phenol;
N²-(4-{[2-(diethylamino)ethyl]oxy}phenyl)-N⁴,5-bis[2-(methyloxy)phenyl]-2,4-pyrimidinediamine;
N²-(4-{[2-(diethylamino)ethyl]oxy}phenyl)-5-(3-furanyl)-N⁴-[2-(methyloxy)phenyl]-2,4-pyrimidinediamine;
1-[5-(2-[(4-{[2-(diethylamino)ethyl]oxy}phenyl)amino]-4-{[2-(methyloxy)phenyl]amino}-5-pyrimidinyl)-2-thienyl]ethanone;
2-(2-[(4-{[2-(diethylamino)ethyl]oxy}phenyl)amino]-4-{[2-(methyloxy)phenyl]amino}-6-pyrimidinyl)phenol;
N²-(4-{[2-(diethylamino)ethyl]oxy}phenyl)-5-(3-fluorophenyl)-N⁴-[2-(methyloxy)phenyl]-2,4-pyrimidinediamine;
[3-(2-[(4-{[2-(diethylamino)ethyl]oxy}phenyl)amino]-4-{[2-(methyloxy)phenyl]amino}-5-pyrimidinyl)phenyl]methanol;
5-(1,3-benzodioxol-5-yl)-N²-(4-{[2-(diethylamino)ethyl]oxy}phenyl)-N⁴-[2-(methyloxy)phenyl]-2,4-pyrimidinediamine;
5-(1-benzothien-3-yl)-N²-(4-{[2-(diethylamino)ethyl]oxy}phenyl)-N⁴-[2-(methyloxy)phenyl]-2,4-pyrimidinediamine;
N⁴-[2-(methyloxy)phenyl]-N²-{3-[2-(4-morpholinyl)ethyl]phenyl}-5,5′-bipyrimidine-2,4-diamine;
N-(1-methylpropyl)-2-[(5-(1H-pyrazol-4-yl)-2-{[4-(1H-1,2,4-triazol-1-ylmethyl)phenyl]amino}-4-pyrimidinyl)amino]benzamide;
N⁴-[2-(3-fluorophenyl)ethyl]-N²-[4-(1H-1,2,4-triazol-1-ylmethyl)phenyl]-5,5′-bipyrimidine-2,4-diamine;
N²-(4-{[2-(diethylamino)ethyl]oxy}phenyl)-5-(3,4-difluorophenyl)-N⁴-[2-(methyloxy)phenyl]-2,4-pyrimidinediamine;
N²-(4-{[2-(diethylamino)ethyl]oxy}phenyl)-5-(2-fluorophenyl)-N⁴-[2-(methyloxy)phenyl]-2,4-pyrimidinediamine;
1-[4-(2-[(4-{[2-(diethylamino)ethyl]oxy}phenyl)amino]-4-{[2-(methyloxy)phenyl]amino}-5-pyrimidinyl)phenyl]ethanone;
N²-(4-{[2-(diethylamino)ethyl]oxy}phenyl)-N⁴-[2-(methyloxy)phenyl]-5-phenyl-2,4-pyrimidinediamine;
4-(2-[(4-{[2-(diethylamino)ethyl]oxy}phenyl)amino]-4-{[2-(methyloxy)phenyl]amino}-5-pyrimidinyl)benzoic acid;
4-(2-[(4-{[2-(diethylamino)ethyl]oxy}phenyl)amino]-4-{[2-(methyloxy)phenyl]amino}-5-pyrimidinyl)benzoic acid;
N⁴-[2-(methyloxy)phenyl]-N²-[4-(1H-1,2,4-triazol-1-ylmethyl)phenyl]-5,5′-bipyrimidine-2,4-diamine;
N²-(4-{[2-(diethylamino)ethyl]oxy}phenyl)-N⁴,5-bis[2-(methyloxy)phenyl]-2,4-pyrimidinediamine;
5-(3-aminophenyl)-N²-(4-{[2-(diethylamino)ethyl]oxy}phenyl)-N⁴-[2-(methyloxy)phenyl]-2,4-pyrimidinediamine;
4-(2-[(4-{[2-(diethylamino)ethyl]oxy}phenyl)amino]-4-{[2-(methyloxy)phenyl]amino}-5-pyrimidinyl)benzaldehyde;
N²-(4-{[2-(diethylamino)ethyl]oxy}phenyl)-N⁴-[2-(methyloxy)phenyl]-5-(2-methylphenyl)-2,4-pyrimidinediamine;
5-(3,4-dichlorophenyl)-N²-(4-{[2-(diethylamino)ethyl]oxy}phenyl)-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)benzonitrile;
N²-(4-{[2-(diethylamino)ethyl]oxy}phenyl)-N⁴-[2-(methyloxy)phenyl]-5-[3-(methyloxy)phenyl]-2,4-pyrimidinediamine;
N²-(4-{[2-(diethylamino)ethyl]oxy}phenyl)-N⁴-[2-(methyloxy)phenyl]-5-[4-(methyloxy)phenyl]-2,4-pyrimidinediamine;
N-(1-methylpropyl)-2-[(2-{[4-(1H-1,2,4-triazol-1-ylmethyl)phenyl]amino}-5,5′-bipyrimidin-4-yl)amino]benzamide;
2-(diethylamino)ethyl {4-[(4-{[2-(methyloxy)phenyl]amino}-5,5′-bipyrimidin-2-yl)amino]phenyl}carbamate;
3-({2-[(4-{[2-(diethylamino)ethyl]oxy}phenyl)amino]-5,5′-bipyrimidin-4-yl}amino)benzonitrile;
N²-(4-{[2-(diethylamino)ethyl]oxy}phenyl)-N⁴-[2-(methyloxy)phenyl]-5-(3-methylphenyl)-2,4-pyrimidinediamine;
N²-(4-{[2-(diethylamino)ethyl]oxy}phenyl)-2′,4′-bis(methyloxy)-N⁴-[2-(methyloxy)phenyl]-5,5′-bipyrimidine-2,4-diamine;
N²-(4-{[2-(diethylamino)ethyl]oxy}phenyl)-N⁴-[2-(methyloxy)phenyl]-5-[1-(2-methylpropyl)-1H-pyrazol-4-yl]-2,4-pyrimidinediamine;
N²-(4-{[2-(diethylamino)ethyl]oxy}phenyl)-N⁴-[2-(methyloxy)phenyl]-5-[2-(methylthio)phenyl]-2,4-pyrimidinediamine;
N-[4-(2-[(4-{[2-(diethylamino)ethyl]oxy}phenyl)amino]-4-{[2-(methyloxy)phenyl]amino}-5-pyrimidinyl)phenyl]acetamide;
5-[2,4-bis(methyloxy)phenyl]-N²-(4-{[2-(diethylamino)ethyl]oxy}phenyl)-N⁴-[2-(methyloxy)phenyl]-2,4-pyrimidinediamine;
N⁴-[3-(2-methyl-1,3-thiazol-5-yl)phenyl]-N²-[4-(1H-1,2,4-triazol-1-ylmethyl)phenyl]-5,5′-bipyrimidine-2,4-diamine;
N⁴-[3-(2-methyl-1,3-thiazol-5-yl)phenyl]-N²-{3-[2-(4-morpholinyl)ethyl]phenyl}-5,5′-bipyrimidine-2,4-diamine;
N²-(4-{[2-(diethylamino)ethyl]oxy}phenyl)-5-(4-ethenylphenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(4-methylphenyl)-2,4-pyrimidinediamine;
N²-(4-{[2-(diethylamino)ethyl]oxy}phenyl)-5-[1-(3-methylbutyl)-1H-pyrazol-4-yl]-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(1H-pyrrolo[2,3-b]pyridin-4-yl)-2,4-pyrimidinediamine;
5-(3-chloro-4-fluorophenyl)-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(8-quinolinyl)-2,4-pyrimidinediamine;
N²-(4-{[2-(diethylamino)ethyl]oxy}phenyl)-5-(4-ethylphenyl)-N⁴-[2-(methyloxy)phenyl]-2,4-pyrimidinediamine;
3-[(2-{[4-(1H-1,2,4-triazol-1-ylmethyl)phenyl]amino}-5,5′-bipyrimidin-4-yl)amino]benzonitrile;
N²-(4-{[2-(diethylamino)ethyl]oxy}phenyl)-N⁴-[2-(methyloxy)phenyl]-5-(2-naphthalenyl)-2,4-pyrimidinediamine;
N²-(4-{[2-(diethylamino)ethyl]oxy}phenyl)-5-[3,5-dimethyl-4-(methyloxy)phenyl]-N⁴-[2-(methyloxy)phenyl]-2,4-pyrimidinediamine;
3-(2-[(4-{[2-(diethylamino)ethyl]oxy}phenyl)amino]-4-{[2-(methyloxy)phenyl]amino}-5-pyrimidinyl)benzamide;
5-[3,4-bis(methyloxy)phenyl]-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-5-(2-fluoro-4-biphenylyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[4-(methylthio)phenyl]-2,4-pyrimidinediamine;
5-[5-chloro-2-(methyloxy)phenyl]-N²-(4-{[2-(diethylamino)ethyl]oxy}phenyl)-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-[3-(trifluoromethyl)phenyl]-2,4-pyrimidinediamine;
N²-(4-{[2-(diethylamino)ethyl]oxy}phenyl)-N⁴-[2-(methyloxy)phenyl]-5-(5-quinolinyl)-2,4-pyrimidinediamine;
5-[2,5-bis(methyloxy)phenyl]-N²-(4-{[2-(diethylamino)ethyl]oxy}phenyl)-N⁴-[2-(methyloxy)phenyl]-2,4-pyrimidinediamine;
1-[3-(2-[(4-{[2-(diethylamino)ethyl]oxy}phenyl)amino]-4-{[2-(methyloxy)phenyl]amino}-5-pyrimidinyl)phenyl]ethanone;
N²-(4-{[2-(diethylamino)ethyl]oxy}phenyl)-5-{3-fluoro-4-[(phenylmethyl)oxy]phenyl}-N⁴-[2-(methyloxy)phenyl]-2,4-pyrimidinediamine;
N²-(4-{[2-(diethylamino)ethyl]oxy}phenyl)-N⁴-[2-(methyloxy)phenyl]-5-(6-quinolinyl)-2,4-pyrimidinediamine;
N²-(4-{[2-(diethylamino)ethyl]oxy}phenyl)-N⁴-{[2-(methyloxy)phenyl]methyl}-5-(1H-pyrazol-4-yl)-2,4-pyrimidinediamine;
N²-(4-{[2-(diethylamino)ethyl]oxy}phenyl)-N⁴-[3-(1-piperidinylmethyl)phenyl]-5,5′-bipyrimidine-2,4-diamine;
N²-(4-{[2-(diethylamino)ethyl]oxy}phenyl)-5-[3-(ethyloxy)phenyl]-N⁴-[2-(methyloxy)phenyl]-2,4-pyrimidinediamine;
N²-(4-{[2-(diethylamino)ethyl]oxy}phenyl)-5-[4-(ethyloxy)phenyl]-N⁴-[2-(methyloxy)phenyl]-2,4-pyrimidinediamine;
N²-(4-{[2-(diethylamino)ethyl]oxy}phenyl)-N⁴-[2-(3-fluorophenyl)ethyl]-5,5′-bipyrimidine-2,4-diamine;
N²-(4-{[2(diethylamino)ethyl]oxy}phenyl)-N⁴-{[2(methyloxy)phenyl]methyl}-5,5′-bipyrimidine-2,4-diamine;
3-(2-[(4-{[2-(diethylamino)ethyl]oxy}phenyl)amino]-4-{[2-(methyloxy)phenyl]amino}-5-pyrimidinyl)benzoic acid;
N²-(4-{[2-(diethylamino)ethyl]oxy}phenyl)-N⁴-[2-(methyloxy)phenyl]-5-(1-thianthrenyl)-2,4-pyrimidinediamine;
N²-(4-{[2-(diethylamino)ethyl]oxy}phenyl)-N⁴-[2-(methyloxy)phenyl]-5-[4-(trifluoromethyl)phenyl]-2,4-pyrimidinediamine;
5-(1-benzofuran-2-yl)-N²-(4-{[2-(diethylamino)ethyl]oxy}phenyl)-N⁴-[2-(methyloxy)phenyl]-2,4-pyrimidinediamine;
N²-{3-[2-(4-morpholinyl)ethyl]phenyl}-N⁴-[2-(phenyloxy)phenyl]-2,4-pyrimidinediamine;
N²-(4-{[2-(diethylamino)ethyl]oxy}phenyl)-5-[2-(ethyloxy)phenyl]-N⁴-[2-(methyloxy)phenyl]-2,4-pyrimidinediamine;
N⁴-[2-(3-fluorophenyl)ethyl]-5-(1H-pyrazol-4-yl)-N²-[4-(1H-1,2,4-triazol-1-ylmethyl)phenyl]-2,4-pyrimidinediamine;
N²-(4-{[2-(diethylamino)ethyl]oxy}phenyl)-N⁴-[2-(methyloxy)phenyl]-5-(4-propylphenyl)-2,4-pyrimidinediamine;
N⁴-[(2-aminophenyl)methyl]-N²-(4-{[2-(diethylamino)ethyl]oxy}phenyl)-5,5′-bipyrimidine-2,4-diamine;
N⁴-{[2-(methyloxy)phenyl]methyl}-N²-{3-[2-(4-morpholinyl)ethyl]phenyl}-5,5′-bipyrimidine-2,4-diamine;
5-(2-biphenylyl)-N²-(4-{[2-(diethylamino)ethyl]oxy}phenyl)-N⁴-[2-(methyloxy)phenyl]-2,4-pyrimidinediamine;
5-(2-biphenylyl)-N²-(4-{[2-(diethylamino)ethyl]oxy}phenyl)-N⁴-[2-(methyloxy)phenyl]-2,4-pyrimidinediamine;
N⁴-[2-(3-fluorophenypethyl]-N²-(4-{[(4-methyl-1-piperazinyl)sulfonyl]methyl}phenyl)-5-(1H-pyrazol-4-yl)-2,4-pyrimidinediamine;
5-(3-biphenylyl)-N²-(4-{[2-(diethylamino)ethyl]oxy}phenyl)-N⁴-[2-(methyloxy)phenyl]-2,4-pyrimidinediamine;
N⁴-{[2-(methyloxy)phenyl]methyl}-N²-[4-(1H-1,2,4-triazol-1-ylmethyl)phenyl]-5,5′-bipyrimidine-2,4-diamine;
4-(2-[(4-{[2-(diethylamino)ethyl]oxy}phenyl)amino]-4-{[2-(methyloxy)phenyl]amino}-5-pyrimidinyl)benzonitrile;
methyl 4-(2-[(4-{[2-(diethylamino)ethyl]oxy}phenyl)amino]-4-{[2-(methyloxy)phenyl]amino}-5-pyrimidinyl)benzoate;
methyl 4-(2-[(4-{[2-(diethylamino)ethyl]oxy}phenyl)amino]-4-{[2-(methyloxy)phenyl]amino}-5-pyrimidinyl)benzoate;
5-[3,5-bis(trifluoromethyl)phenyl]-N²-(4-{[2-(diethylamino)ethyl]oxy}phenyl)-N⁴-[2-(methyloxy)phenyl]-2,4-pyrimidinediamine;
N²-(3-{[2-(dimethylamino)ethyl]oxy}phenyl)-N⁴-[2-(methyloxy)phenyl]-5-(1H-pyrazol-4-yl)-2,4-pyrimidinediamine hydrochloride;
and salts thereof.

7. A pharmaceutical composition comprising a therapeutically effective amount of a compound according to claim 1, and one or more of pharmaceutically acceptable carriers, diluents or excipients.

8. A method of treating a disorder in a mammal, said disorder being mediated by inappropriate Wee1 activity, comprising: administering to said mammal a therapeutically effective amount of a compound according to claim 1.

9. A method of treating cancer in a mammal, comprising: administering to said mammal a therapeutically effective amount of a compound according to claim 1.

10-11. (canceled)
